(19) United States
(12) Patent Application Publication
(10) Pub. No.: US 2013/0261599 A1
(43) Pub. Date: Oct. 3, 2013

(54) Dosing Unit With Safety Valve

(71) Applicants: Ulrich Haueter, Grosshoechstetten (CH); Hanspeter Niklaus, Riken (CH); David Teutsch, Schuepfen (CH); Florian Kuehni, Bern (CH)

(72) Inventors: Ulrich Haueter, Grosshoechstetten (CH); Hanspeter Niklaus, Riken (CH); David Teutsch, Schuepfen (CH); Florian Kuehni, Bern (CH)

(73) Assignee: ROCHE DIAGNOSTICS INTERNATIONAL AG, Rotkreuz (CH)

(21) Appl. No.: 13/902,443

(22) Filed: May 24, 2013

Related U.S. Application Data
(63) Continuation of application No. PCT/EP2011/069569, filed on Nov. 7, 2011.

(30) Foreign Application Priority Data
Nov. 25, 2010 (EP) 10192607.9

(51) Int. Cl. A61M 5/168; A61M 5/142

(52) U.S. Cl. CPC A61M 5/16881; A61M 5/14216
USPC 604/506; 604/152

(57) ABSTRACT

A valve for an infusion pump device comprising a pump connection fluidly connected to a piston pump, an infusion site connection fluidly connected to an infusion set, and a reservoir connection fluidly connected to a reservoir is presented. The valve is in a first state (I) when the pump connection is fluidly connected to the infusion site connection and the reservoir connection is sealed closed and in a second state (II) when the pump connection is fluidly connected to the reservoir connection and the infusion site connection is sealed closed. The valve comprises one or more conduits, grooves, and/or recesses connected to environment and arranged so that no path exists within the valve between the infusion site connection and the reservoir connection that does not cross at least one of the conduits, grooves, and/or recesses, establishing a drain, independently if a path is closed during normal operation of the valve.

### CROSS-REFERENCE TO RELATED APPLICATIONS

This application is a continuation of PCT/EP2011/069569, filed Nov. 7, 2011, which is based on and claims priority to EP 10192607.9, filed Nov. 25, 2010, which is hereby incorporated by reference.

### BACKGROUND

The present disclosure generally relates to a system and method for infusion pump devices and, in particular, to valves and dosing units for infusion pump devices and to methods for conveying liquid medication in an infusion pump device.

Devices for the automated release of liquid medications are normally used with patients who have a continuous and, in the course of the day, varying need of a liquid medicine which can be administered by infusion. Specific applications are, for example, certain pain therapies, cancer therapies and the treatment of diabetes mellitus, in which computer controlled infusion pump devices are used. Such devices are particularly useful for ambulatory therapy, and are generally carried attached on, or near, the body of a patient. The medicine reservoir often comprises medicine supply sufficient for one or several days. The liquid medication is supplied to the patient's body from the medicine reservoir through an infusion cannula or an injection needle.

Ambulatory infusion pump devices are typically of the syringe driver type, where the liquid medication to be administered to the patient is stored in a cylindrical glass cartridge or ampoule acting as the reservoir and is conveyed to the body of the patient by displacing a piston within the cylinder. An example of such an infusion pump device is schematically depicted in FIG. 1(a). A cylinder 11 of the dosing unit 1 comprises the complete reservoir 21 of liquid medication of the infusion pump device 2. An outlet 17 is fluidly connected 421 to an infusion tubing 31, which on its other end is fluidly connected to an infusion site interface 33 attached to the body of a patient 9. A piston head 12 arranged in the cylinder is unidirectionally displaced along the cylinder axis by a drive system 14 via a piston shaft or threaded spindle 13. The drive system is controlled by a control unit 22.

A number of drawbacks of such a design are known in the art. In particular, such pump devices have a limited precision, because they involve pumping very small volumes, typically in the nanolitre range, out of a cartridge having an overall volume in the range of millilitre, typically, for example, about 3 ml. Thus, to achieve a precise dosing of the liquid medication, it is necessary to very precisely displace the piston. Already small deviations can lead to over dosing or under dosing. Furthermore the forces needed to actuate the piston are comparably high due to the friction between the walls of the glass cartridge and the sealing of the piston. This leads to demanding requirements for the drive system and the mechanical parts involved, as well as the control unit of the pump. As a consequence such infusion pump devices are expensive.

Another problem is the lower limit of the length of such an infusion pump device. The complete supply of liquid medication has to be stored in the cartridge acting as the pump cylinder. The cross-sectional area of the piston has to be below a certain limit, for precision reasons and in order to limit the device thickness, which is known to be a particularly critical dimension with respect to comfort and discreetness during application. The minimum overall length of the device is then essentially given by the resulting minimum length of the cylinder, which is detrimental to the provision of compact infusion pumps.

Particularly in self-administration of medications, for example insulin, the patients using the medication in question and administering it themselves by an infusion pump are increasingly emphasizing convenience and discretion, which restricts the acceptable size and weight of such devices. Particular the overall length, width and thickness should be as small as possible, in order not be evident through clothing and to be carried as comfortable as possible.

Alternative approaches have been proposed, in which a separate dosing unit is provided downstream from the reservoir. Since the primary reservoir does not have to fulfill additional functions, its dimensions can be optimized in view of the compactness of the infusion pump device. Such a dosing unit can comprise for example a micro membrane pump or a micro piston pump, especially designed for precise metering of small volumes. A piston pump with smaller dimensions retrieves liquid medication from a larger primary reservoir, e.g. a collapsible reservoir, and conveys the liquid medication in a precise manner to the injection site on the body of the patient.

When filled, the cylinder of the piston pump acts as a secondary reservoir, holding a restricted amount of liquid medication. When the cylinder is empty, the piston pump retrieves new liquid medication from the primary reservoir. Such pumps are generally full-stroke pumps, where the cavity of a membrane pump or the cylinder of a piston pump is always completely emptied. Hence the inner volume of the pump must correspond to the smallest volume increment that may have to be delivered, typically in the nanoliter range.

While several designs for such dosing units are known in the art, they are rather complex, expensive and critical with respect to large scale manufacture since they integrate a number of functional components, in particular metering components and valves and are frequently made from materials which are costly and/or critical in production and processing, such as silicon.

A simpler infusion pump device, where check valves, are realized by flexible wings of a plunger arranged in the cylinder of the dosing unit, ensures the correct flow of the liquid medication during the refilling mode and the pumping mode. To ensure user safety, such a design requires a cost intensive drive system, since any uncontrolled activation of the drive system due to a malfunction would inevitably lead to a an overdosing event.

The liquid medications that are administered by liquid infusion pump devices are generally highly effective. The accuracy of the dosing unit is therefore of utmost importance, to avoid any potentially hazardous dosing errors. Such accuracy can be ensured by an appropriate design of the various parts of an infusion pump device, as well as by using high quality components.

As an additional level of safety, the dosing unit can be construed in a way that even the a complete malfunction of one component, for example the drive system, as unlikely as it may be, cannot lead to an overdosing hazard. For that purpose a second component of the device has to intervene. Infusion pump devices with such a design are known from the prior art. In one variant of such an infusion pump device, a 4/3 or 3/3 way valve is arranged at a front end of the cylinder of a dosing unit, as schematically shown in FIG. 1(b).

A piston 12, 13 arranged in the cylinder of the dosing unit 1 can be bidirectionally displaced along the cylinder axis by a drive system. In a first state of the valve 4, an inlet conduit 18 fluidly connected to the primary reservoir 21 is fluidly connected to the cylinder and an outlet conduit 17 fluidly connected to the infusing tubing is disconnected from the dosing unit. This state of the valve is applied during the refill mode, when the dosing unit retracts the piston and sucks liquid medication from the primary reservoir 21 into the cylinder.

In a second state of the valve, as it is shown in FIG. 1(b), both the inlet 18 and the outlet 17 are disconnected from the dosing unit 1. This blocked state of the valve is applied when, for example, the infusion tubing has to be temporarily disconnected from the infusion pump device.

In a third state of the valve, the cylinder of the dosing unit is fluidly connected to the outlet conduit 17, thereby establishing a fluid connection to the body of the patient 9. The inlet conduit 18 is disconnected from the dosing unit. This third valve state is applied during the pumping mode, when liquid medication is conveyed from the secondary reservoir 16 in the cylinder of the dosing unit to the subcutaneous tissue of the patient.

During application, the dosing unit is in the third state most of the time. For refilling, it is in the first state for a time span in a range of several seconds to maximum of several minutes, depending on design parameters such as the cylinder volume and the displacement speed of the piston. The second state is passed only when switching between the first state and the third state. In typical embodiments, the system is such that only the first state and the third state can be moved into in a defined way.

Therefore, there is a need to provide an advantageous dosing unit for use in an infusion pump device with increased safety, which especially prevents an uncontrolled flow of medication to the patient in case of a valve defect, allows a precise dosing of liquid medication, is reliable, producible with high quality at low costs in a large-scale manufacture and that can function in any orientation in space.

### SUMMARY

According to the present disclosure, a dosing unit for an infusion pump device and a method for its use is presented. The dosing unit comprises a piston pump, a reservoir connector fluidly connected to a reservoir, an infusion site connector fluidly connected to an infusion set, and a valve. The valve comprises a valve seat and a valve member that are rotatable to each other, a pump port fluidly connected to the piston pump, an upstream port fluidly connected to the reservoir connector, and a downstream port fluidly connected to the infusion site connector. The valve can be in a downstream state corresponding to a specific angular orientation of valve seat and valve member, where the pump port is fluidly connected to the downstream port and the upstream port is sealed closed; in a venting state corresponding to one or more specific angular orientations of valve seat and valve member where the downstream port and the upstream port are sealed closed and the pump port is fluidly connected to a venting chamber or to atmosphere; and in an upstream state corresponding to a specific angular orientation of valve seat and valve member where the pump port is fluidly connected to the upstream port and the downstream port is sealed closed. Upon rotation of the valve member in regard to the valve seat from the downstream state to the upstream state and/or from the upstream state to the downstream state, the valve passes at least one angular orientation of valve seat and valve member that corresponds to the venting state.

Accordingly, it is a feature of the embodiments of the present disclosure to provide an advantageous dosing unit for use in an infusion pump device with increased safety, which especially prevents an uncontrolled flow of medication to the patient in case of a valve defect, allows a precise dosing of liquid medication, is reliable, producible with high quality at low costs in a large-scale manufacture and that can function in any orientation in space. Other features of the embodiments of the present disclosure will be apparent in light of the description of the disclosure embodied herein.

### BRIEF DESCRIPTION OF THE SEVERAL VIEWS OF THE DRAWINGS

The following detailed description of specific embodiments of the present disclosure can be best understood when read in conjunction with the following drawings, where like structure is indicated with like reference numerals and in which:

FIGS. 1a-b illustrate schematically two infusion pump devices according to the prior art.

FIGS. 2a-b illustrate schematically (a) an infusion pump device with a dosing unit and (b) a schematic view of the valve of the dosing unit according to an embodiment of the present disclosure.

FIGS. 3a-b illustrate a combined pump cylinder and valve of a dosing unit, (a) in an isometric view, (b) in a longitudinal section along plane A-A, and (c) in a longitudinal section along plane B-B according to an embodiment of the present disclosure.

FIGS. 4a-c illustrate the pump cylinder and valve seat in FIG. 3, (a) in an isometric view, (b) in a front view onto the valve seat, and (c) in a longitudinal section along plane A-A according to an embodiment of the present disclosure.

FIGS. 5a-c illustrate the outer component of the valve member in FIG. 3, (a) in an isometric view, (b) in a longitudinal section along plane A-A, and (c) in a longitudinal section along plane B-B according to an embodiment of the present disclosure.

FIGS. 6a-b illustrate the inner sealing component of the valve member in FIG. 3, (a) in an isometric view, and (b) in a longitudinal section along plane A-A according to an embodiment of the present disclosure.

FIGS. 7a-c illustrate different views of a valve member of a dosing unit in frontal view onto the sealing area according to an embodiment of the present disclosure.

FIGS. 8a-d illustrate a dosing unit with radially mounted valve member, (a) in a view onto the front end of the cylinder along the longitudinal axis, (b) in a longitudinal section along plane A-A, (c) in a cross section along plane B-B, the valve being in a first state, and (d) in a longitudinal section along plane C-C, the valve being in a second state according to an embodiment of the present disclosure.

FIGS. 9a-g illustrate a dosing unit with axially mounted valve member, (a) in a view onto the front end of the cylinder along the longitudinal axis, (b) in a cross sectional view along plane A-A, (c) in a cross section along plane B-B, the valve being in state I, (d) showing a detail view, (e) in an isometric view of the valve member, (f) in a cross section through the valve member alone, along plane C-C, and (g) in a isometric view of the cylinder and the valve seat alone according to an embodiment of the present disclosure.

FIGS. 10a-b illustrate schematically a valve similar to FIG. 9, with a relief area, in which the sealing elements of the valve member are relied from mechanical stress according to an embodiment of the present disclosure.

FIGS. 11a-c illustrate three variants of a valve member for use in a dosing unit similar to the ones in FIGS. 9 and 10 according to an embodiment of the present disclosure.

FIG. 12 illustrates a valve with three different conduits connectable to the cylinder according to an embodiment of the present disclosure.

FIGS. 13a-c illustrate a dosing unit with separate valve seat and cylinder, (a) in a longitudinal section, (b) in a longitudinal section along plane A-A, and (c) in an isometric view according to an embodiment of the present disclosure.

FIGS. 14a-h illustrate a dosing unit where an open cylinder is closed on one end by the valve member according to an embodiment of the present disclosure.

FIGS. 15a-b illustrate a dosing unit with a valve member in the form of a valve disc, (a) in a longitudinal section, and (b) in a top view on the valve disc alone according to an embodiment of the present disclosure.

FIGS. 16a-b illustrate a variant of the valve in FIG. 15, (a) in a longitudinal section, and (b) in a top view on valve disc alone according to an embodiment of the present disclosure.

FIG. 17a-d illustrate a valve that is particularly suitable for long time storage prior to use, (a) in a side view of the valve in state I, (b) in a cross-section of FIG. 17(a) along plane A-A, (c) in a side view of the valve in storage mode, and (d) in a cross-section of FIG. 17(c) along plane A-A according to an embodiment of the present disclosure.

### DETAILED DESCRIPTION

In the following detailed description of the embodiments, reference is made to the accompanying drawings that form a part hereof, and in which are shown by way of illustration, and not by way of limitation, specific embodiments in which the disclosure may be practiced. It is to be understood that other embodiments may be utilized and that logical, mechanical and electrical changes may be made without departing from the spirit and scope of the present disclosure.

A piston pump based dosing unit with a valve system is presented. The valve, a 3/3 or 4/3 way valve, can be realized as a rotatable cylinder head acting as a valve member, which can interact with a fixed cylinder tube, acting as the valve seat. Alternatively, a rotatable cylinder can act as the valve member, mounted in a fixed valve seat. The actuator of the piston can indirectly actuate the valve member by rotating the cylinder, which can be frictionally connected to the piston.

This design can generally provide a high safety level. However, in the generally unlikely but well possible case of both valves showing a leakage, caused, for example, by a problem in the manufacture, the cylinder may be bridged, resulting in a direct fluidic connection between the inlet and the outlet.

The basic principle of a dosing unit can be the insertion of an additional venting mode between the refilling mode of the dosing unit, where the pump cylinder can be fluidly connected with the primary reservoir holding the liquid medication supply, and the pump mode, where the pump cylinder can be fluidly connected to the infusion set, or the patient respectively. In this additional venting mode the pump cylinder can be temporarily connected to environment.

The environment can be a special venting chamber, for example, the inner, empty volume of the device housing. The environment can be a hermetically sealed space, for example, hermetically sealed device housing. In such a case, the environmental pressure can be the specific pressure inside the device housing. If the environment is connected to the surrounding atmosphere, it can have atmospheric pressure. The environment can for example be inner volume of a compartment of device housing, such as, hermetically sealed device housing.

Venting can allow the equalization of a positive or negative pressure difference between the pump cylinder and the environment. The venting mode can be passed each time when the dosing unit switches between refilling mode and pumping mode. Thus each switching between the refilling mode and the pump mode can include pressure equalization of the pump cylinder. Any pressure difference that may be present in the primary reservoir, caused, for example, by high or low temperatures and/or mechanical stress exerted onto the primary reservoir can be equalized. This can prevent the unintentional and uncontrolled administration of medication due to overpressure in the cylinder, as well as the unintentional and uncontrolled retraction of fluid from the infusion set into the cylinder. This advantageous function scheme with venting mode can be realized by a specially designed valve arranged between the pump cylinder, the primary reservoir, and the infusion set.

The valve can be designed such that the switching process does not lead to the shifting even of small amounts liquid within the fluid system. This measure can further increase the accuracy of a dosing unit. This can be favourably achieved via a rotational rather than a linear valve motion.

A basic embodiment of a dosing unit for an infusion pump device can comprise a piston pump, a reservoir connector fluidly connected to a reservoir, an infusion site connector fluidly connected to an infusion set, and a valve. The valve can comprise a valve seat and a valve member that are rotatable to each other, a pump port fluidly connected to the piston pump, an upstream port fluidly connected to the reservoir connector, and a downstream port fluidly connected to the infusion site connector. The valve can be in a downstream state, corresponding to a specific angular orientation of valve seat and valve member, where the pump port is fluidly connected to the downstream port and the upstream port is sealed closed; in a venting state, corresponding to one or more specific angular orientations of valve seat and valve member, where the downstream port and the upstream port are sealed closed and the pump port is fluidly connected to a venting chamber or to atmosphere; and in an upstream state, corresponding to a specific angular orientation of valve seat and valve member where the pump port is fluidly connected to the upstream port, and the downstream port is sealed closed. Upon rotation of the valve member in regard to the valve seat from the downstream state to the upstream state and/or from the upstream state to the downstream state, the valve can pass at least one angular orientation of valve seat and valve member that corresponds to the venting state.

Thus whenever the valve switches between the upstream state and the downstream state, the pump cylinder can be temporarily fluidly connected to the venting chamber or to atmosphere allowing equalization of a positive or negative pressure difference between the pump cylinder and the environment.

In one embodiment, the dosing can have one or more venting passages arranged on the valve seat and/or the valve member. The one or more venting passages can be fluidly connected to the venting chamber or to atmosphere. In the latter case, the one or more venting passages can be connected to atmospheric pressure. The venting passages can provide a flow path that can have a fluidic resistance toward environment that is low, particularly when compared to a closed valve path. The venting passages can be, for example, conduits, grooves, and/or recesses.

In one embodiment of the dosing unit, one or more of the venting passages can be arranged on the valve seat and/or the valve member in such a way that any possible geometrical path between the upstream port and the downstream port in a gap formed between abutting surfaces of the valve seat and the valve member can cross at least one of the venting passages. In the venting state, the pump port can be fluidly connected to one or more of the venting passages.

In another embodiment of a dosing unit, the valve can pass an intermediate state, where all ports can be sealed closed when switching between the upstream state and the venting state and/or when switching between the venting state and the downstream state.

In yet another embodiment of a dosing unit, the valve seat can comprise at least one passage conduit fluidly connected to the piston pump and the valve member can comprise a first connection conduit fluidly connected to the infusion site connector and a second connection conduit fluidly connected to the reservoir connector. The fluid connection between a passage conduit and one of the connection conduits can be established when an opening of the passage conduit overlaps with an opening of the corresponding connection conduit.

In another embodiment, such a valve can comprise one or more venting conduits fluidly connectable to a passage conduit. Such a valve can comprise limitations to the valve seat and the valve member rotational displacement in regard to each other. One limit of rotational displacement can correspond to the downstream state of the valve and the other limit of rotational displacement can correspond to the upstream state of the valve. The venting state of the valve can correspond to one or more rotational displacements between the two maximum displacements.

The valve can fluidly connect in the downstream state to a cylinder of the piston pump with an outlet conduit fluidly connected to the infusion site connector and in the upstream state with an inlet conduit fluidly connected to the reservoir connector.

In another embodiment of the dosing unit for an infusion pump device can comprise a piston pump a reservoir connector fluidly connected to a reservoir, an infusion site connector fluidly connected to an infusion set, and a valve. The valve can comprises a valve seat and a valve member that can be rotatable to each other, a pump port fluidly connected to the piston pump, an upstream port fluidly connected to the reservoir connector, and a downstream port fluidly connected to the infusion site connector. The valve can be in a downstream state, where the pump port is fluidly connected to the downstream port and the upstream port is sealed closed; and in an upstream state, where the pump port is fluidly connected to the upstream port and the downstream port is sealed closed. One or more venting passages can be arranged on the valve seat and/or the valve member. The one or more venting passages can be fluidly connected to a venting chamber or to atmosphere and can be arranged in such a way that any possible geometrical path between the upstream port and the downstream port in a gap formed between abutting surfaces of the valve seat and the valve member can cross at least one of the venting passages.

In such a dosing unit, any liquid in the case of valve leakage or a defect or malfunctioning valve may otherwise directly flow from the primary reservoir to the infusion set via a shortcut, thus circumventing the pump cylinder, can come across one or more venting passages connected to environment. Accordingly, the liquid can leave the fluid system via these venting passages toward the environment.

In one embodiment of this dosing unit, the valve can be in a downstream state, where the pump port is fluidly connected to the downstream port and the upstream port is sealed closed; in a venting state, where the downstream port and the upstream port are sealed closed and the pump port is fluidly connected to a venting chamber or to atmosphere; and in an upstream state, where the pump port is fluidly connected to the upstream port and the downstream port is sealed closed. Upon rotation of the valve member in regard to the valve seat from the downstream state to the upstream state and/or from the upstream state to the downstream state, the valve can pass at least one angular orientation of valve seat and valve member that corresponds to the venting state. The valve can pass an intermediate state, where all ports are sealed closed, when switching between the upstream state and the venting state, and/or when switching between the venting state and the downstream state. In the venting state, the pump port can be fluidly connected to one or more of the venting passages.

In another embodiment of a dosing unit, the valve seat can comprise at least one passage conduit that is fluidly connected to the piston pump and the valve member can comprise a first connection conduit fluidly connected to the infusion site connector and a second connection conduit fluidly connected to the reservoir connector. The fluid connection between a passage conduit and one of the connection conduits can be established when an opening of the passage conduit overlaps with an opening of the corresponding connection conduit.

In another embodiment, the valve can comprise one or more venting conduits fluidly connectable to a passage conduit. The valve of such a dosing unit can comprise limits on the valve seat and the valve member that limit the rotational displacement in regard to each other. One limit of rotational displacement can correspond to the downstream state of the valve and the other limit of rotational displacement can correspond to the upstream state of the valve. The venting state of the valve can correspond to one or more rotational displacements between the two maximum displacements.

The valve can be fluidly connect in the downstream state to a cylinder of the piston pump with an outlet conduit fluidly connected to the infusion site connector and in the upstream state with an inlet conduit fluidly connected to the reservoir connector. In a dosing unit, either the valve seat or the valve member can be an integral part of the cylinder. The rotation axis of the valve seat and the valve member can be collinear to the longitudinal axis of the cylinder.

An infusion pump device can comprise a dosing unit. Such an infusion pump device can comprise a reservoir fluidly connected to the reservoir connector of the dosing unit, and/or an infusion set fluidly connected to the infusion site connector of the dosing unit.

A method for safely conveying a liquid medication in an infusion pump device can comprise the steps of: a) providing an infusion pump device with a dosing with a reservoir fluidly connected to the reservoir connector of the dosing unit, and/or an infusion set fluidly connected to the infusion site connector of the dosing unit; b) switching the valve of the dosing unit to the venting state; c) switching the valve of the dosing unit to the upstream state; d) conveying a certain amount of liquid medication from the reservoir to the pump cylinder of the piston pump, by generating a negative pressure in the pump cylinder; e) switching the valve of the dosing unit to the venting state; f) switching the valve of the dosing unit to the downstream state; g) conveying a certain amount of liquid medication, in one or more portions, from the pump cylinder to the infusion site connector, by generating a positive pressure in the pump cylinder; and h) repeating steps b) to g).

In an embodiment of the method, after pumping each single portion of the liquid medication toward the infusion site interface, the pump cylinder can be disconnected from the infusion site interface.

One part of the valve remaining static in regard to the cylinder can be the valve seat, while the other part of the valve, which is displaced in regard to the first part during use, can be the valve member. This nomenclature, however, is only chosen as a convention and is not intended to limit the invention. Particularly, it can be the valve seat that is rotated in regard to the structure of the infusion pump device, while the valve member remains static in regard to the structure. Thus the terms valve seat and valve member can be exchangeable.

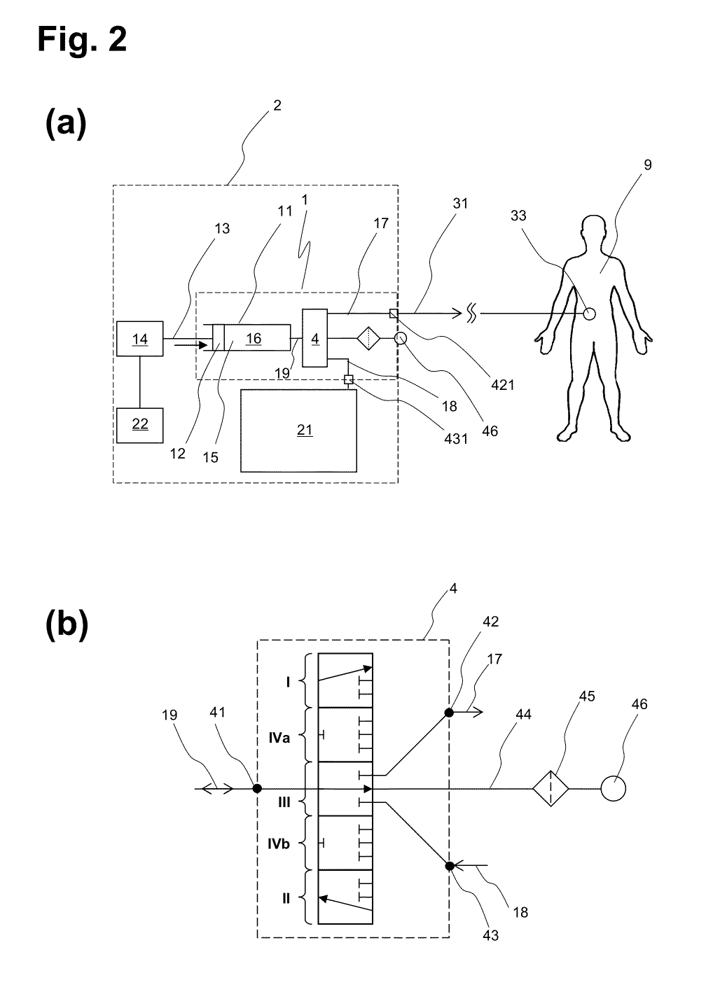

Referring initially to FIG. 2, a schematic view of an embodiment of an infusion pump device is shown in FIG. 2(a). The infusion pump device 2 can comprise a primary reservoir 21, a dosing unit 1, and a control unit 22. The primary reservoir 21 can hold the supply of liquid medication, for example, an insulin solution.

The primary reservoir 21 can be a fully, or partially, collapsible container, thus its content cannot be pressurized in regard to environmental pressure. Suitable containers for that purpose are for example known from EP 2193815 and EP 2179755, the disclosure of which is hereby incorporated by reference. Instead of a flexible reservoir 21, a rigid ampoule or cartridge can be used. The pressure equalization can then be achieved with a venting conduit 44, or a freely displaceable piston. In the latter case, the piston may be subject to a biasing spring force, in order to overcome friction between piston and cartridge.

The dosing unit 1 can comprise a piston pump with a pump cylinder 11 and a piston head 12 slidably arranged within the cylinder 11 and sealed closing the cylinder 11, thereby defining an inner volume 15 of the pump cylinder 11. The piston head 12 can be actuated by a drive system 14, for example, by coupling a piston shaft 13 with the drive system 14. The drive system 14, and thus the dose of administered medication, can be controlled by a control unit 22 of the infusion pump device 2.

The dosing unit 1 can retrieve liquid medication from the primary reservoir 21 via inlet conduit 18 and can pump the liquid medication in small, accurate doses via outlet conduit 17, infusion tubing 31 and an infusion cannula of an injection site interface 33 into the body of the patient 9. The infusion tubing 31 can be fluidly coupled to the outlet conduit 17 with a suitable coupling unit 421.

Alternatively, the infusion tubing 31 may be omitted. In such an embodiment, the whole infusion pump device 2 can be directly located at the infusion site and can be attached to the body 9, e.g., via an adhesive pad.

The correct flow of the liquid medication within the infusion pump device 2 can be controlled by valve 4 (shown in more detail in FIG. 2(b)). The valve 4 can be a 4/5 way valve having five distinct states I, IVa, III, IVb, II that can be switched in sequential order. Pump port 41 of the valve 4 can be fluidly connected to the inner volume 15 of the pump cylinder 11 via passage conduit 19. Downstream port 42 can be fluidly connected with outlet conduit 17. Upstream port 43 can be fluidly connected to inlet conduit 18.

In active state I of the valve 4, pump port 41 can be interconnected to downstream port 42, thereby establishing a fluid path between the inner volume 16 of the pump cylinder 11, the passage conduit 19, the valve 4, the outlet conduit 17, and the tubing coupling 421, and from there to the infusion site interface 33. State I can be applied during the pump mode of the dosing unit 1 when the piston 12 is displaced into the cylinder 11 and liquid medication in the cylinder 11 is expelled through the fluid path toward the patient 9.

For refilling the secondary reservoir 16 of the dosing unit 1, the valve 4 can be switched to active state II, where upstream port 43 is interconnected to pump port 41, thereby establishing a fluid path between the primary reservoir 21 and the inner volume 1 of the pump cylinder 11, via the inlet conduit 18, the valve 4, and the passage conduit 19. The dosing unit 1 can now be in its refilling mode.

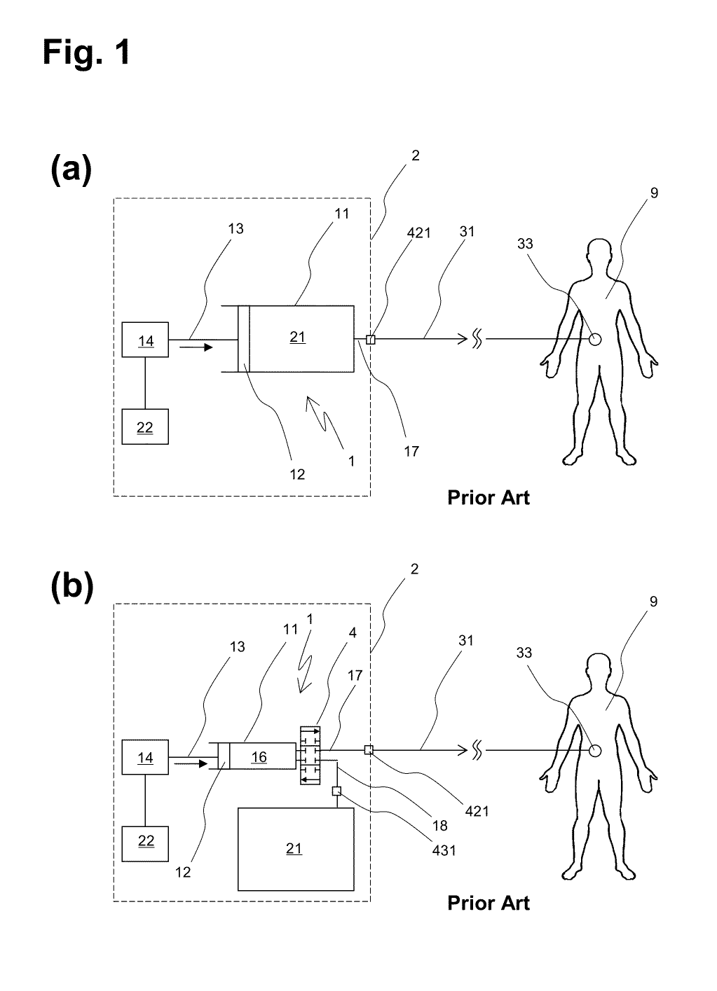

For switching the valve 4 between state I and state II, the valve 4 can switch to an intermediate state, where all ports 41, 42, 43 are completely disconnected, to ensure that no unwanted connection can temporarily exist during the transition from one active state to another. This is known from the prior art (see e.g. FIG. 1(b)). In the valve 4 in FIG. 2(b) these intermediate states can correspond to states IVa and IVb.

In FIG. 2(b), the valve 4 as used in a dosing unit 1 can have an additional active state III, to which the valve 4 can switch between the intermediate states IVa and IVb. In this state III, the inner volume 16 of the cylinder 11 can be fluidly interconnected to environment 46, via passage conduit 19, valve 4, and venting conduit 44. The dosing unit 1 can be in the venting phase.

This additional state III can provide advantages over the prior art. For example, the pressure differential between the inside 16 of the pump cylinder 11 and the environment 46 can be essentially zero, except when displacing the piston 12 in either of the refill mode or the pump mode, where the pressure differential can convey the liquid within the fluid system. If a user changes his location between two dosing events, for example during travel, the changing atmospheric pressure can lead to a positive or negative pressure differential between the pump cylinder 11 and the surrounding atmosphere. In FIG. 2(b), an additional sterile filter 45 can be arranged in the venting conduit 44, which may, however, not be necessary.

A substantive over-pressure in the cylinder 11 may arise when filling the cylinder 11 with the valve 4 being in state I. If a substantive over-pressure is present in the primary reservoir 21, for example, due to thermal expansion and/or mechanical pressure exerted onto the reservoir 21, this over-pressure can be transferred to the cylinder 11. When a prior art valve subsequently switches back from state I to state II via an intermediate break state, this results in the over-pressure being relieved by an undesired administration of liquid to the patient 9. In the worst case, this can lead to a potentially hazardous overdosing event. In a dosing unit of the present disclosure, however, this can be prevented by passing state III in between. Any pressure differential that may potentially exist between the inner volume 16 of the pump cylinder 11 and the environment 46 can automatically be equalized through venting conduit 44, when the dosing unit 1 is temporarily in the venting mode and the valve 4 is in state III. This can be an additional advantageous effect.

Another advantage of such a design can be the additional level of safety provided. If in a prior art dosing unit, one component fails, for example if the drive system has a malfunction, this malfunction does not result in a potentially hazardous event, since the valve 4 will provide a barrier in the fluid system. Only if both components fail, a potentially hazardous event can take place. A valve 4 can now provide an additional level of safety.

No fluid connection can be established due to a failure of a valve 4 between the primary reservoir 21 and the outlet conduit 17, since the liquid stream can have to pass the opening toward the venting conduit 44 and can flow out of the venting conduit. Thus the venting conduit 44 can add an additional level of isolation between the outlet conduit 17 and the dosing pump and the primary reservoir 21.

In the schematic depiction of the valve 4 in FIG. 2(b), the different active and intermediate states of the valve 4 are shown as a linear shift mechanism. In another embodiment of the valve 4, the valve 4 can be switched in a rotational motion.

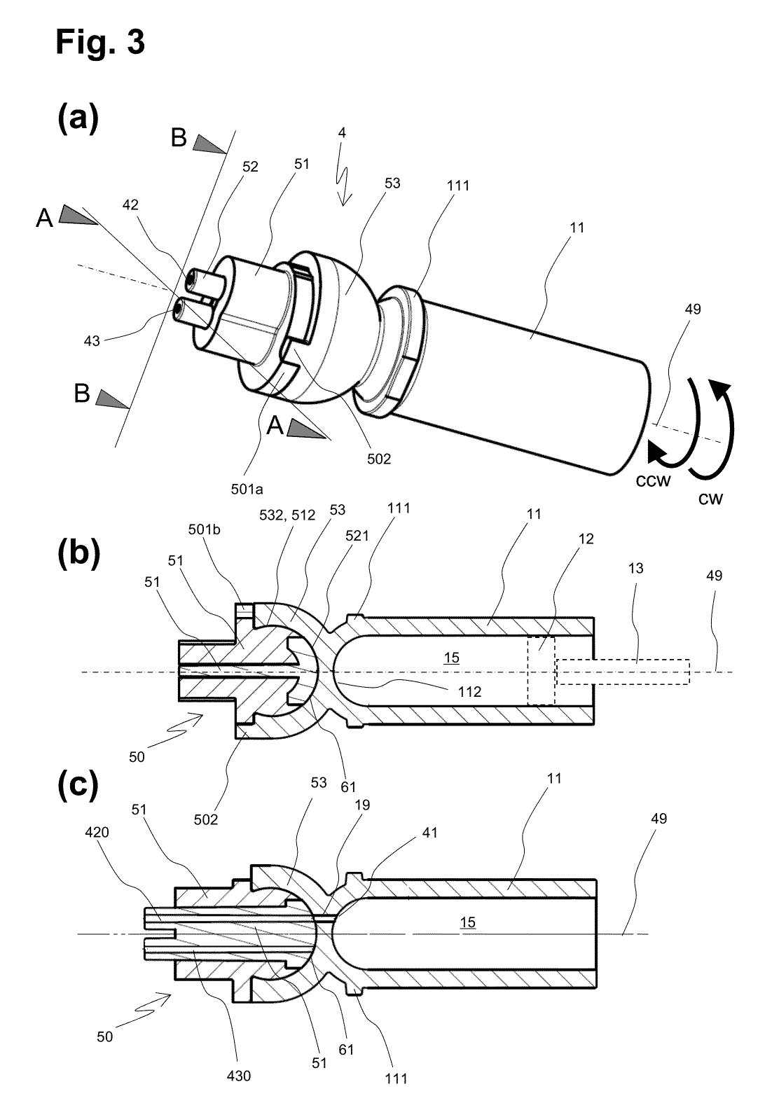

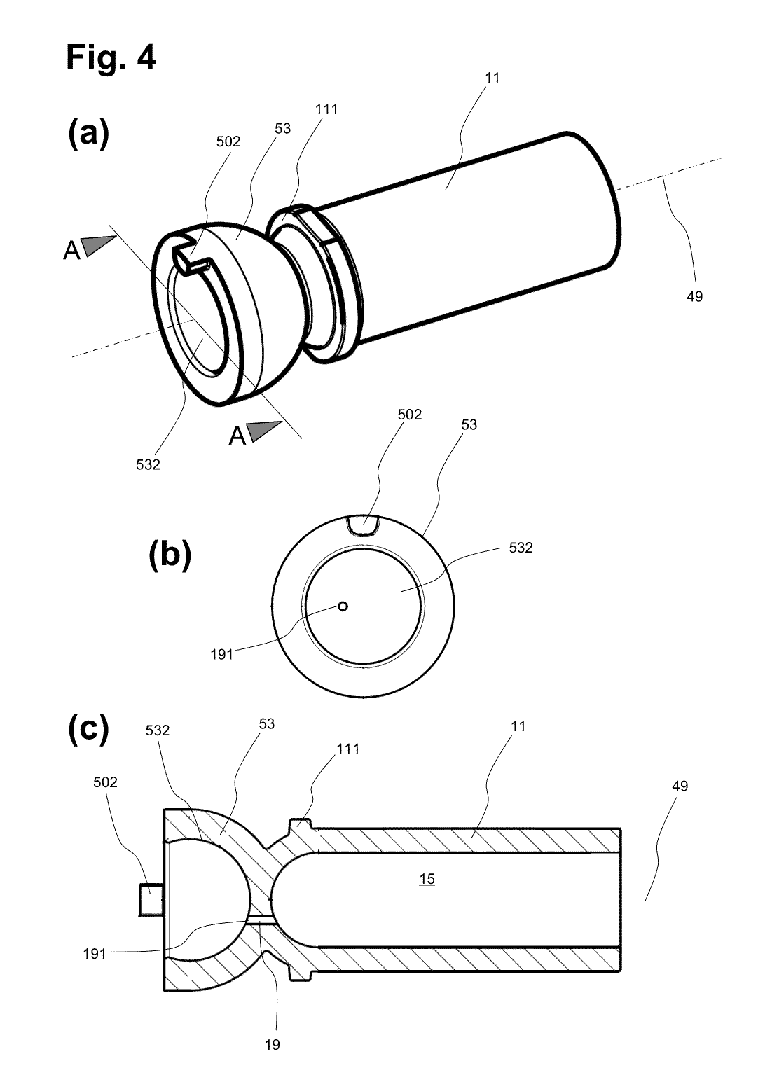

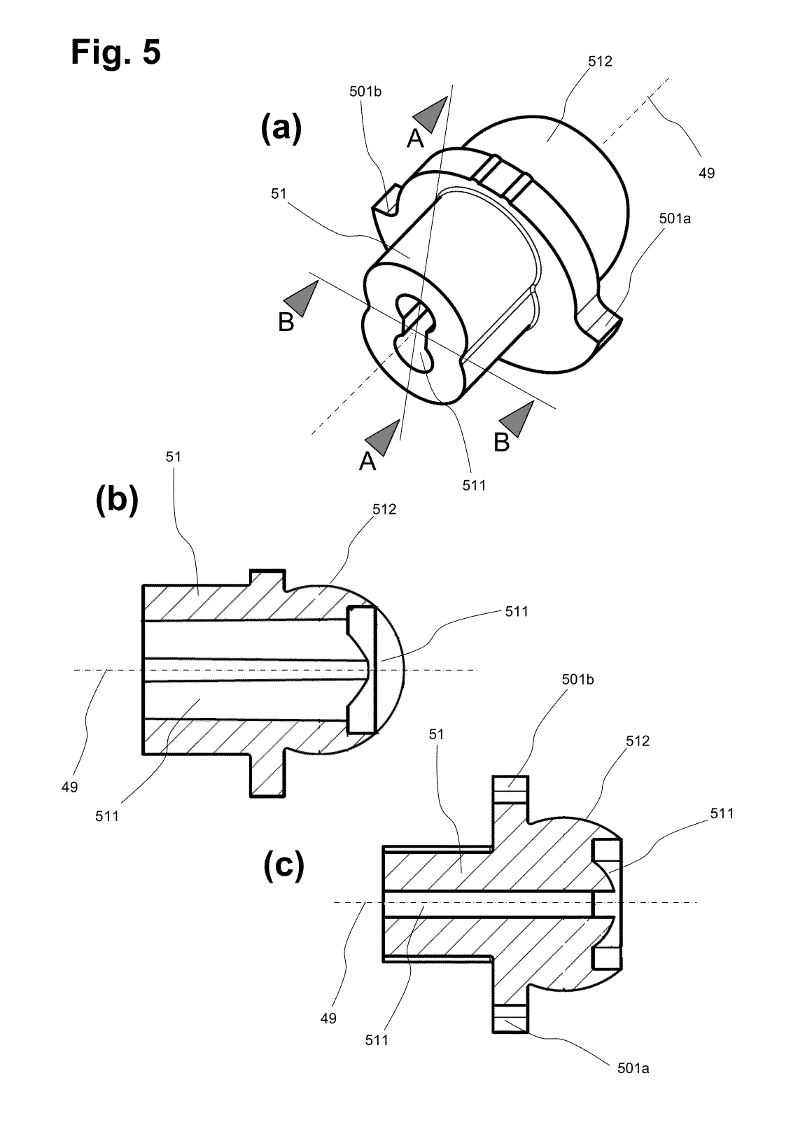

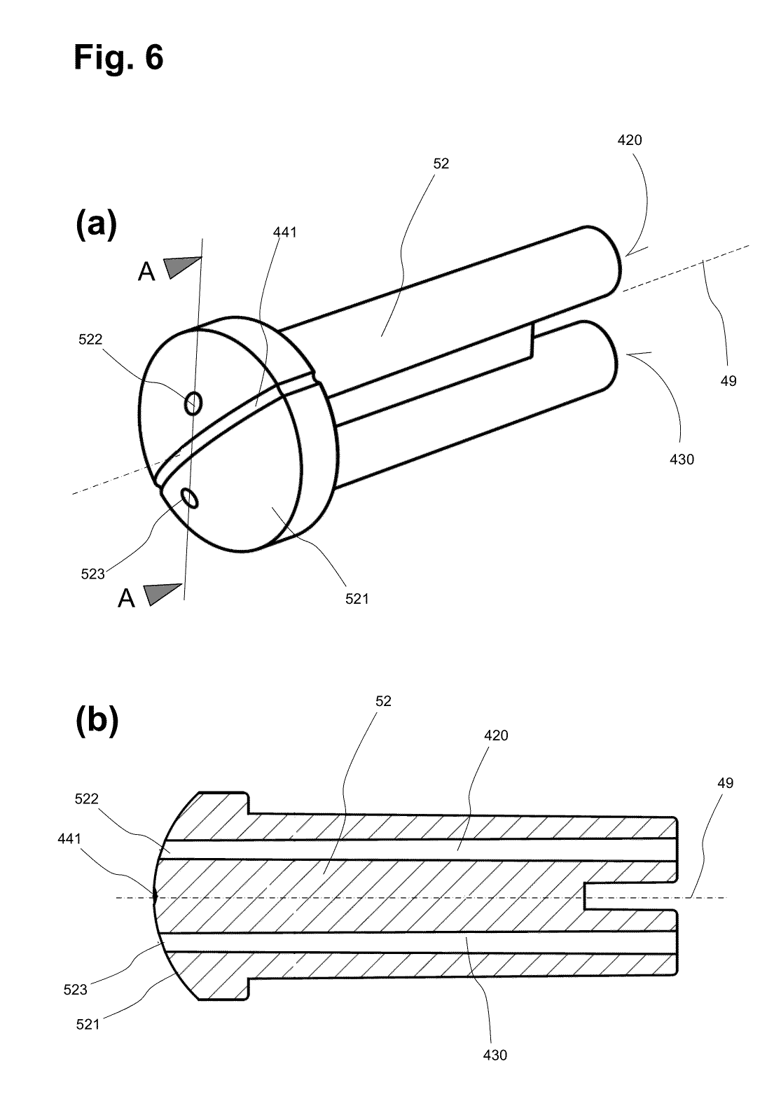

An embodiment of a combined pump cylinder 11 and valve 4 of a dosing unit 1 is shown in FIGS. 3 to 6. While FIG. 6 shows a combined pump cylinder 11 and valve 4, FIG. 4 shows the pump cylinder 11 with integral valve seat 53 and FIGS. 3 and 5 show the two components 51, 52 of a disassembled valve member 50.

The dosing unit 1 can comprise a piston pump with a cylinder 11 and a piston 12, 13 (schematically shown with dashed lines) and a valve 4 arranged at the end of the cylinder 11 opposite to the piston. The valve 4 can comprise a valve seat 53 and a valve member 50. The valve seat 53 can be an integral part of the cylinder 11, which can be manufactured by injection molding. The cylinder 11 can be rotatably mounted in the dosing unit 1 having a guiding ring 111 mounted in a corresponding bearing structure (not shown) of the dosing unit 1.

A passage conduit 19 can be between the inner volume 15 of the cylinder 11 and a concave holding structure 532 of the valve seat 53. The passage 19 can be offset from the longitudinal axis 49 of the cylinder 11 and valve 4. The concave structure 532 can interact with a shape-matched, spherically shaped counterpart 512 of the valve member 50, which can be rotatably mounted within structure 532. Instead of the spherical shape of the interacting surfaces 512, 532, any other rotationally symmetric shape can also be applied. The concave structure 532 can be undercut, in order to positively lock the valve member 50 in a longitudinal direction in the valve seat 53. During assembly, the member seat can be snapped over the valve member 50.

The valve member 50 can be rotationally fixed in regard to the longitudinal axis 49. The valve member 50 can comprise a first, outer component 51 and a second, inner component 52, which can be arranged in a corresponding cavity 511 of the outer component 51. The outer component 51 can be made from a rigid polymer material so that friction between valve seat 53 and valve member 50 is minimal. The inner component 52 may provide a certain elasticity, since it can act as the sealing 521 of the valve 4, sealed connecting the opening 192 of the passage conduit 19 and the openings 522, 523 of the connection conduits 420, 430.

The valve member 50 can be manufactured by two component injection molding. For the outer component 51, a rigid thermoplastic polymer material can be used that is acceptable for medical use, for example, Acrylnitril-Butadien-Styrene (ABS), Polyamide (PA), or Polycarbonate (PC). The same rigid materials can be used for the valve seat 53 and the cylinder 11. For the inner component 52, a comparably soft thermoplastic material can be used, for example thermoelastic polymers of the TPE-V (cross-linked olefins), TPE-S (styrene block copolymers), TPE-U (urethane) class.

The inner component 52 can comprise two connection conduits 420, 430 arranged parallel to the longitudinal axis. They can open toward openings 522, 523 on the sealing area 521 of the inner component 52, which can form part of the spherical surface 512 of the valve member 50. The position of the two openings 522, 523 can be chosen so that the opening 192 of the passage conduit 19 can overlap with one opening 522 when the cylinder 11 is in a first rotational orientation (corresponding to valve state I) and can overlap with the other opening 523 when the cylinder 11 is in a second angular orientation (corresponding to a valve state II). When two openings overlap, a fluid connection between the passage conduit 19 and one of the connector conduits 420, 430 can be established. The other conduit 430, 420 can be sealed closed.

The parameters of the material for the sealing area 521 and the valve member 50, as well as the dimensions of the parts, can be chosen so that in the intermediate states, where the openings do not overlap, the conduits 19, 420, 430 can be sealed closed. As can be seen in FIG. 6(a), a venting groove 441 can be diagonally arranged across the sealing area 521. The venting groove can be fluidly connected to environment pressure via a venting conduit (not shown).

When switching the valve 4 between the two active states I and II, the valve member 50 and the cylinder 11 with its integral valve seat 53 can be rotated in regard to each other along axis 49. In one embodiment, the cylinder 11 can be rotated by about 180° while the valve member 50 is fixed.

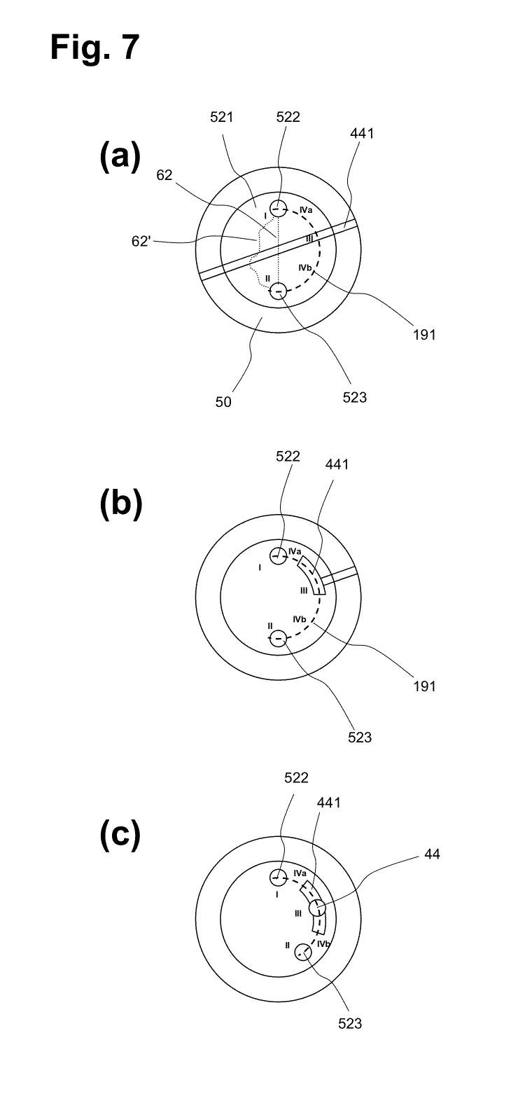

When the cylinder 11 is rotated, the opening 192 of the passage conduit 19 can travel in regard to surface 521 of the valve member 50 on a circular path 191. Different angles of rotation, such as, for example, 90° may be used as well. This is schematically shown in FIG. 7(a), where a front view of a valve member 50 is shown. The path 191 of the opening 192 is shown as a dashed line. When the opening 192 lies on opening 522, the valve 4 can be in active state I. Upon rotation of the cylinder 11, the opening 192 can travel along the path passing intermediate break state IVa, before reaching the venting groove 441. When the opening 192 overlaps with the venting groove 441, the cylinder 11 can be fluidly connected with environment pressure 46 and the valve 4 can be in active state III. The opening 192 can then pass the second intermediate break state IVb and can finally reach the active second state II with openings 192, 523 overlapping.

During the pumping mode and refilling mode, the valve 4 can remain a comparably long time in the two states I and II, the time period in which the valve 4 remains in state III can be comparably short and can depend on the rotational motion of the cylinder 11. If a constant angular velocity of the rotation is applied, the length of the venting mode period can depend on the angular velocity, the radius of the circular path 191, as well as the diameter of the opening 192 and the width of the venting groove 441. In one embodiment, the time for switching between state I and state II can lie in the range of several seconds.

To increase the time period in which venting of the cylinder 11 can take place, the rotation of the cylinder 11 may be slowed down, the rotation may be temporarily halted in the state III, or the width of the venting groove 441 may be adjusted. Such measures, however, may not be necessary.

In FIG. 6(a), and FIG. 7(a), the venting groove 441 can cross the complete sealing area 521. Such a design can have the advantage that the effective diameter of the venting conduit 44 can be larger and that there can exist two venting pathways. Another advantage can be the fact that in the case of a malfunction of the valve 4, no geometrical fluid path 62, 62′ between the inlet conduit 18 and the outlet conduit 17 can exist that does not cross the venting conduit 44. Any liquid unintentionally leaving the primary reservoir 21 can drain via the venting groove 441 and the venting conduit 44 and cannot enter the outlet conduit 17.

For some designs, the dosing unit 1 may be stored long-term in state III, resulting in the sealings being in a relieved, stress-free state.

Various other designs of the sealing area and the arrangement of the openings 522, 523 and the venting groove 441 can be possible. Two examples are shown in FIGS. 7(b) and (c). In FIG. 7(b), the venting groove 441 can comprise a radial segment arranged on the path 191, thereby increasing the length of the venting mode period when the switching rotation takes place at a constant velocity. In FIG. 7(c), the venting conduit 44 can directly open toward the sealing surface 521 and can be combined with a segmental venting groove 441. In this embodiment, the two openings 522 523 of the connection conduits 420, 430 cannot be arranged on opposite ends, but closer on the switching path 191. Thus, the switching angle can be smaller than 180°. Similarly, the switching angle may be larger than 180°.

To switch the valve 4 between the different states, the cylinder 11 with integral valve seat 53 can be rotated in regard to the fixedly mounted valve member 50. This may for example be achieved by a separate actuator arranged to rotate the cylinder 11 around its longitudinal axis 49. However, in order to provide a reliable and cost efficient dosing unit, the number of separate components and systems should be as few as possible. In one embodiment, the valve 4 switching mechanism can be combined with the piston displacement mechanism of the dosing unit 1 (not shown). For that purpose, the end of the cylinder 11 opposite to the valve seat 53 can be provided with a threaded nut 115, which can interact with a threaded portion of the piston shaft 13. The piston shaft 13 can be connected to the drive system 14 in such a way that it can be rotated both clockwise (cw) and counterclockwise (ccw), while at the same time being displaceable along axis 49.

In FIGS. 3(a) to (c), the valve 4 can be in state I, with the inner volume 15 connected to conduit 420. A cam 502 of the valve seat 53 can be in contact with a stopper 501a of the valve member 50. A further rotation of the cylinder 11 in the counterclockwise direction can be blocked. In state I, the dosing unit 1 can be in its pumping mode. By counterclockwisely rotating the threaded piston shaft 13, which can be mounted in the threaded nut 115 of the cylinder 11, the piston can be shifted toward the front end 112 of the cylinder 11. The inner volume 15 can be decreased and liquid medication in the cylinder 11 can be expelled toward the patient.

Changing the dosing unit 1 from pump mode to refill mode can be achieved by simply reversing the rotation direction of the piston shaft 13 from counterclockwise to clockwise. The friction between the threaded shaft 13 and the threaded nut 115 as well as between the cylinder 11 (guiding ring 111, concave surface 532 of valve seat) and the corresponding fixed counterparts (guiding ring bearing, surface 512 of valve member) can be balanced so that the frictional force acting between shaft 13 and cylinder 11 can be larger than the frictional force acting between cylinder 11 and bearing 47. As a result, the cylinder 11 can be frictionally coupled to the shaft 13 rotating in the clockwise direction and can also rotate in direction B. The cam 502 can wander along the circumference of the valve member 50 and can finally arrive at the second stopper 501a. The cylinder 11 cannot rotate any further and the cylinder 11 can be frictionally decoupled from the still rotating shaft 13. The valve 4 can now be in state II with the primary reservoir 21 and connection conduit 430 connected to the inner volume 15.

The dosing unit 1 may be designed so that the cylinder 11 and the piston can move synchronously, that is, without any relative motion for this rotation. In this way, dosing errors that may otherwise result from a residual relative motion between plunger and cylinder 11 during the switching can be prevented. This can be advantage, because the dosing errors resulting from several switching operations would otherwise sum up over the usage time of a dosing cylinder 11 and a primary reservoir 21. Besides pure friction coupling, any other suitable coupling arrangements may be applied.

The piston shaft 13 can continue to rotate in a clockwise direction, which can result in a displacement of the shaft 13 and the connected piston head 12 out of the cylinder 11, thereby increasing the inner volume 15 and sucking liquid medication from the primary reservoir 21 into the cylinder 11.

Changing the dosing unit 1 back to pumping mode, the rotation of the shaft 13 can be changed back to counterclockwise. The cylinder 11 can then turn from state II to state I, passing state III in between, where the freshly refilled dosing cylinder 11 can be vented to environment pressure 46. After the cam 502 has reached the first stopper 501a, the shaft 13 can continue to rotate counterclockwise and the piston can start moving inward expelling liquid medication through conduit 420. In one embodiment, the time in the refill mode can be in the range of about a minute.

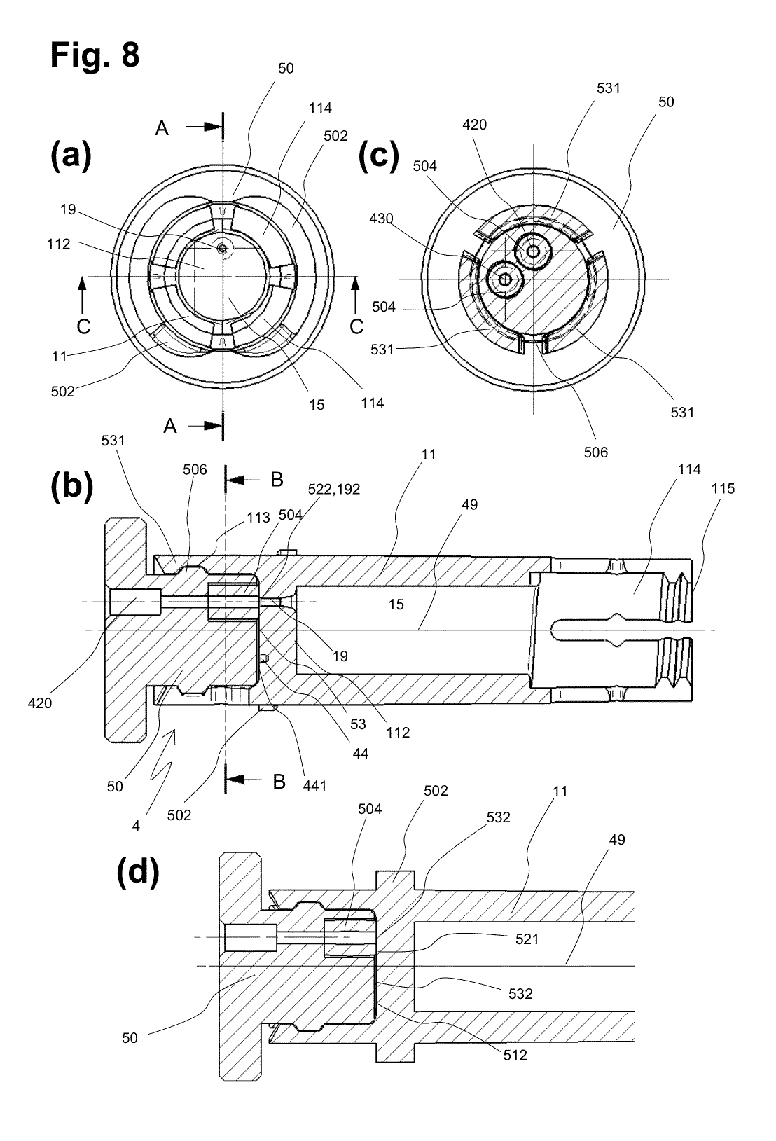

Another embodiment of a dosing unit 1 is disclosed in FIG. 8, with a valve member 50 that can be radially mounted in the valve seat 53. The valve seat 53 can be an integral part of the cylinder 11 and can comprise three segment-shaped snap fingers 531 with a circumferential bearing groove 113 to lock onto a guide ring 506 of the valve member 50. Thus the valve member 50 can be positively locked in the longitudinal direction 49, while freely rotatable around the axis 49. A passage conduit 19 can be in the front wall of the cylinder 11 between the cylinder front 112 and the planar front surface 532 of the valve seat 53 facing toward the valve member 50.

The valve seat 53, valve member 50, and cylinder 11 can be made from a rigid polymer material as discussed above.

At the back end of the cylinder 11, a threaded nut 115 can interact with a threaded portion of the piston shaft (not shown). In one embodiment, the back end of the cylinder 11 with the threaded nut 115 can be divided into four snap finger elements 114, which can allow a simple assembly of the components of the piston pump.

The valve member 50 can comprise two longitudinal conduits 420, 430 arranged at an angular distance of about 90°. At the surface 512 facing toward the valve seat 53, cylindrical sealing elements 504 can be in the conduits 420, 430. In the assembled valve 4, the surfaces 512, 532 cannot be in contact, thereby establishing a thin venting gap 441, which can be fluidly connected to environment pressure 46 via a venting conduit 44 opening toward the valve seat surface 532, as well as the slots between the snap fingers 531.

The sealing elements 504, which can be made from a suitable thermoelastomeric material as discussed above, can slightly protrude from the surface 512 of the valve member 50 so that in the assembled valve 4, they can be pressed against the valve seat surface 532 and seal the conduit 420, 430 against the venting gap 441.

FIG. 8(b) shows the valve 4 in state I with the conduit 420 connected to passage conduit 19 and conduit 430 (not shown) sealed closed. When switching the valve 4, the cylinder 11 with integral valve seat 53 can be rotated with respect to the valve member 50 around the longitudinal axis by about 90° arriving at state II. The other conduit 430 can now be fluidly connected to passage conduit 19 and conduit 420 can be sealed closed, as shown in FIG. 8(d). The rotation of the cylinder 11 can be actuated as discussed in FIGS. 3 to 6. To limit the 90° motion of the cylinder 11, two circumferential cam elements 502 can be on the cylinder 11 which can interact with stopper elements of the dosing unit 1 (not shown).

As discussed for the valve 4 in FIG. 8, the intermediate valve stages IVa, IVb can correspond to the situations where the opening 192 of the passage conduit 19 is sealed covered by the sealing rim 521 of one of the sealing elements 504.

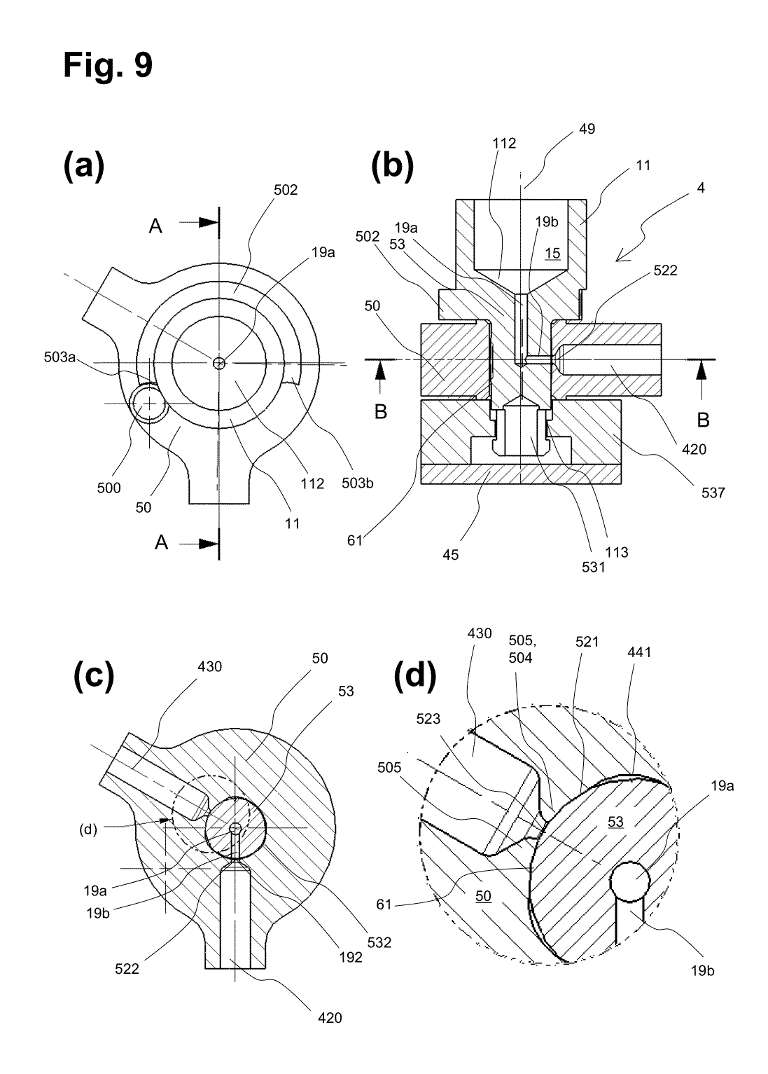

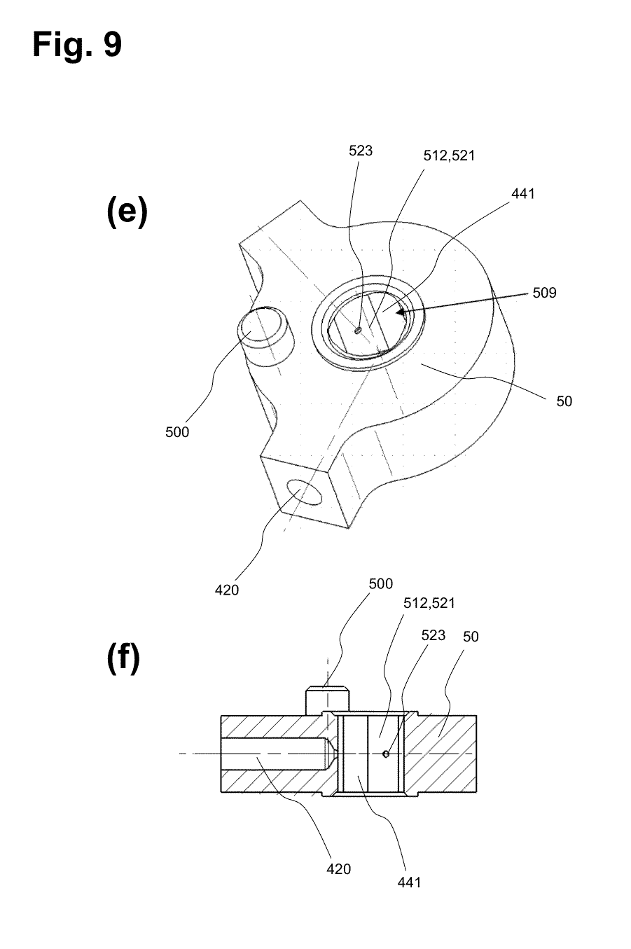

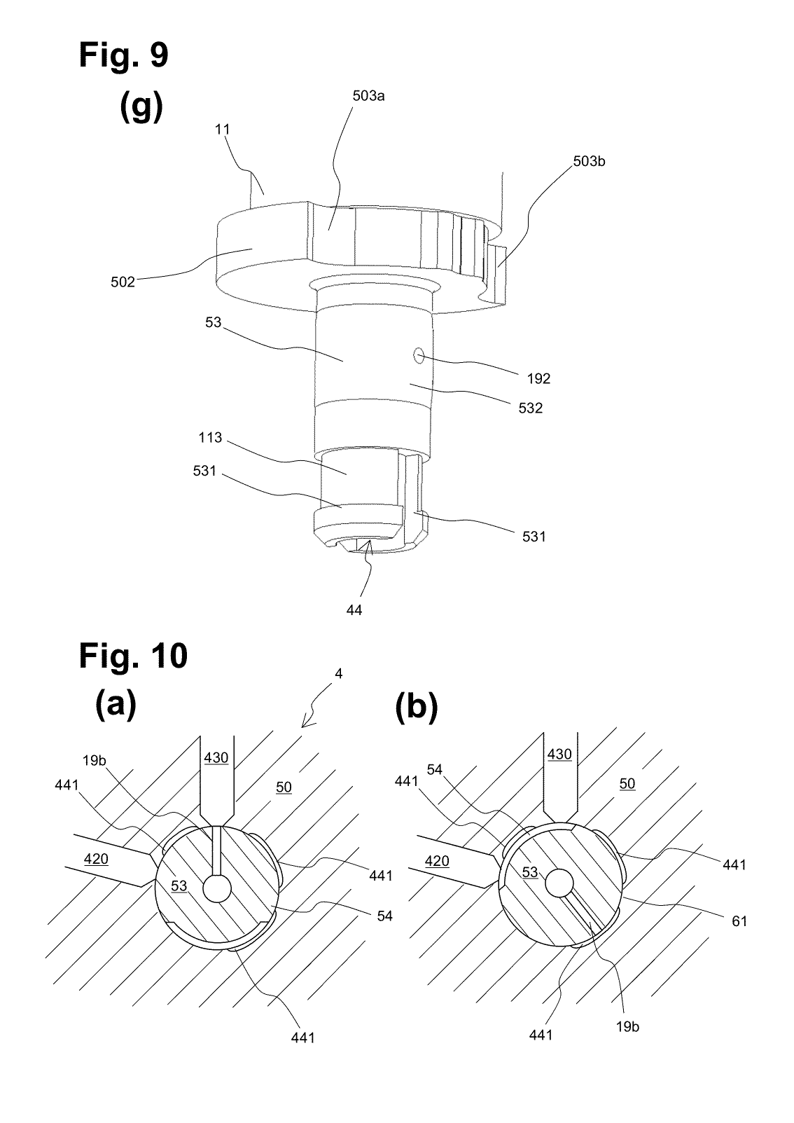

Another embodiment of a dosing unit 1 is shown in FIG. 9. In this embodiment, the valve 4 can comprise a valve seat 53 realized as a central, cylindrically shaped valve core oriented parallel to the longitudinal axis 49. A valve member 50 can be rotatably mounted on the valve seat 53. The valve seat 53 can have two snap fingers 531, which lock with an opening of a locking disk 537, thereby positively locking the valve member 50 in longitudinal direction.

The valve member 50 can have the shape of an oblate cylinder 11 with two protruding arms comprising the connection conduits 420, 430 and a central cylindrical bore for the valve core 53. The conduits 420, 430 can open toward the bore 509 at an angular distance of about 120°. Arranged between the two openings 522, 523, a shallow venting recess 441 in the bore face can be provided (state III of the valve 4) which can be connected to venting conduit 44 formed by the space between the snap fingers 531 and the bore of the locking disk 537. The locking disk 537 can be covered with a sterile filter 45 which can protect the valve 4 from dirt and biological contamination. For that purpose, the locking disk 537 can be connected with the valve seat 53 in such a way that the only connection to the environment can be by the sterile filter 45. The sterile filter 45 can adsorb any liquid that may leak from the valve 4 though the venting conduit 44.

In the shown embodiment, a second venting recess 441 can be provided in an angular position behind opening 523, the purpose of which can be to not vent the cylinder 11, since opening 192 cannot reach the second venting recess 441, but to provide a barrier between conduit 420 from conduit 430 in case of a valve malfunction. Thus any liquid stream in both directions around the valve core can reach a venting recess 441 before coming close to another conduit and can drain through that recess 441.

In one embodiment, the surface of the venting recesses or grooves can have a special structure and/or hygroscopic coating that can improve the wetting of the surface and thus can accelerate the drain of any liquid reaching the recess 441.

The valve seat 53 can comprise a passage conduit with two portions 19a, 19b. A first portion 19a can exit from the front wall 192 of the cylinder 11 along the central axis after an approximate 90° turn, can continue as second portion 19b, and can open toward opening 192 on the cylinder face. The openings 522, 523 of the conduits 420, 430 can be arranged so that they can overlap with opening 192 when the valve 4 is in the corresponding rotational position. The shown embodiment may not comprise additional sealing elements sealed connecting the overlapping conduits 19, 420, 430, which can be achieved by using comparably rigid materials for the valve seat 53 and member 50 and choosing the dimensions so that a certain compression of the material can take place in the sealing areas 521.

As in the previously discussed embodiments, the limit stop of the two states I and II can be realized by cams and stoppers. A single cam 500 can be provided on the valve member 50, which can be fixedly mounted in the dosing unit 1. A segmental cam 502 of the valve seat 53 can provide two stoppers 503a, 503b interacting with the cam 500 of the valve seat 53 and delimiting the rotational motion of the rotatably mounted cylinder 11 and valve seat 53.

FIG. 9 shows the valve 4 in state I with the conduit 420 fluidly connected to the cylinder 11 via passage conduit 19a, 19b. The opening 523 of the other conduit 430 can be sealed closed by the surface 532 of the valve core, thereby disconnecting conduit 430 from the fluid system.

An advantage of the design of the opening, as shown in FIG. 9(d), can be the comparably thin wall 505 surrounding the openings 522, 523. By choosing the radius of the valve core slightly larger than the radius of the bore 509 of valve member 50, the surrounding wall 505 can be compressed and can act as a circular sealing lip 504 around the opening. The sealing effect can even be increased when the hydrostatic pressure in a conduit increases since the walls 505 can be pressed against the valve core surface 532 due to hydrostatic force resulting from the pressure difference. This can be relevant for the primary reservoir 21 connected 18 to connection conduit 430.

The continued elastic deformation of the circular wall 505 used in a valve 4 as discussed above to seal the fluid connection between conduits 19b and 420/430 can lead to creep deformation of both the wall 505 and the counter-surface 532 of the valve seat 53, which can be detrimental for the quality of the valve 4 and thus may need to be minimized. This can be important if the dosing unit 1 is intended for a long operational lifetime. But also for dosing units designed only for a comparably short life, time creep may be minimized since the shelf time prior to purchase of such a unit may need to be as long as possible. In one embodiment, this goal can be achieved by providing a relief recess on the valve seat 53. In a parking position of the valve 4, both sealing lips 521, which can be subject to mechanical shear stress during operation, can be located above the recess, where they can be relieved from shear stress. Thus in this parking position, no creep can take place. Such a parking position can always be chosen when the dosing unit 1 is not connected to a primary reservoir 21 and/or an infusion set 31.

One embodiment of such a valve 4 in a dosing unit 1 is schematically shown in FIG. 10, where (a) shows the valve 4 in active state II with conduit 430 fluidly connected to conduit 19b and conduit 420 sealed closed and (b) shows the valve 4 in the parking position with both sealing lips 505 located over the relief recess 54 while the opening 192 can be located over a venting recess 441.

The relief recess 54 which can have a connection to the venting conduit 44 can be realized as a segmental recess 441 on the cylinder face of the valve seat 53. Since during normal operation the relief recess 441 cannot be connected to any of the conduits 420, 430, the maximum angular distance of the two conduits on the circumference of the bore 509 can be limited. In one example, the angular distance between the two conduits can be slightly less than 90° while the recess 54 can span over slightly over 90°.

An alternative approach to increase the sealing in a valve 4 with a cylindrical valve core can be to adapt the shape of the valve core instead of the cylindrical bore 509. In such an embodiment, the valve core 53 at the position of the opening 192 of the passage conduit may have a slightly larger radius, which can be compressed in the valve states I and II, thereby improving the sealing. To avoid creeping, a relief recess 441 in the face of the bore 509 can be provided, which can park the valve core in a position where it may not be subject to mechanical stress. Instead of a dedicated relief recess, a venting recess can be used.

To avoid any incorrect use of a valve 4 unit, the operation of the valve 4 can be mechanically prevented while in the parking position.

Further embodiments of valve members are shown in FIG. 11. In FIG. 11(a), the openings 522, 523 can be arranged opposite to each other and four venting recesses 441 can be arranged on the bore surface, while FIG. 11(c) depicts rectangularly arranged conduits. In FIG. 11(c), the coaxial conduits 420, 430 can be offset from the rotation center.

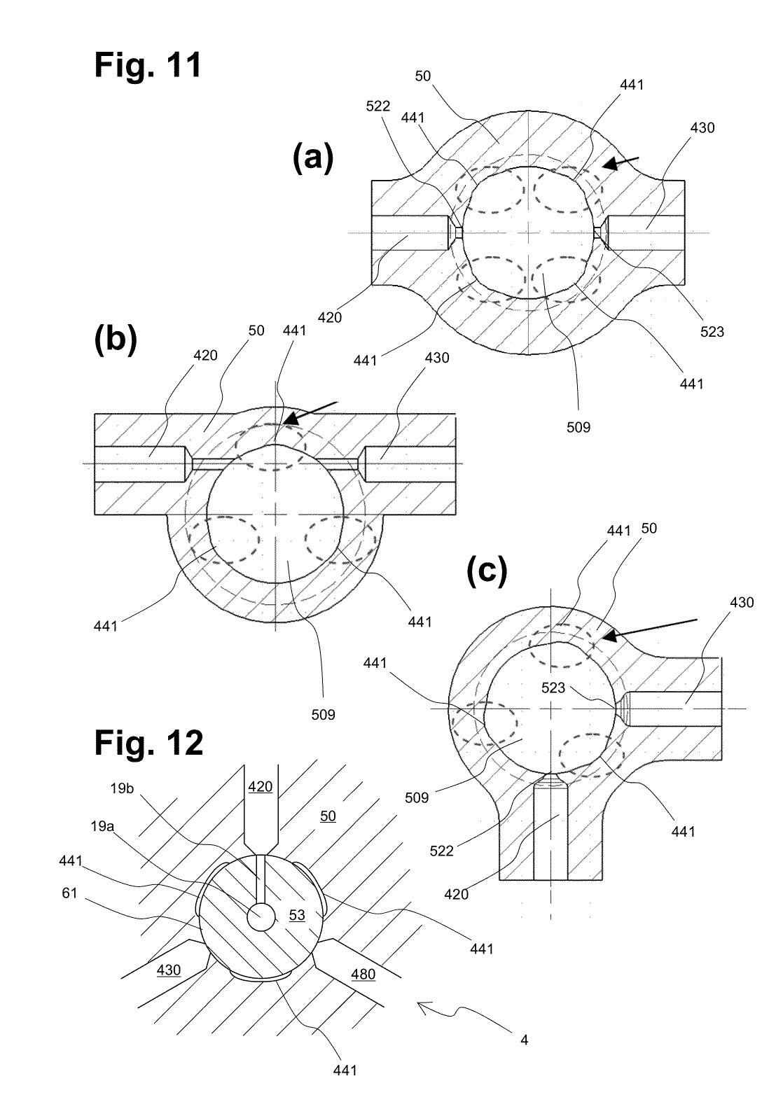

FIG. 12 depicts an embodiment of a valve 4 with three different conduits 420, 430, 480 connectable to the cylinder 11. An additional connection, either to an additional primary reservoir 21 or to an additional infusion set 31 can be an advantage. The additional connection to the primary reservoir 21 can be advantageous since such a dosing unit 1 can for example allow easy administration of different liquid medications (for example two different insulin products with a fast and a slow effectiveness profile). It can also be possible to use two primary reservoirs with the same medication which then can allow the exchange of one primary reservoir 21 or the refilling of that reservoir 21 even in the refilling mode.

Another advantageous application of such a valve 4 can be the possibility to use the dosing pump for refilling the primary reservoir 21 from an external reservoir, for example a standard vial containing liquid medication or a cartridge intended to be used in another type of device. In such a case, the external reservoir can be fluidly connected to the reservoir conduit 480 and the liquid medication can be conveyed toward the primary reservoir 21 by repeated actuation of the pump cylinder 11. Such a possibility can also allow realizing a disposable unit including a dosing unit 1 and an initially empty primary reservoir 21 without the need of providing a separate mechanism for filling/refilling the reservoir. Such a disposable unit can be used together with a reusable unit comprising all the other elements of the infusion pump device 2 that do not come into contact with liquid medication and thus can have a longer operational lifetime.

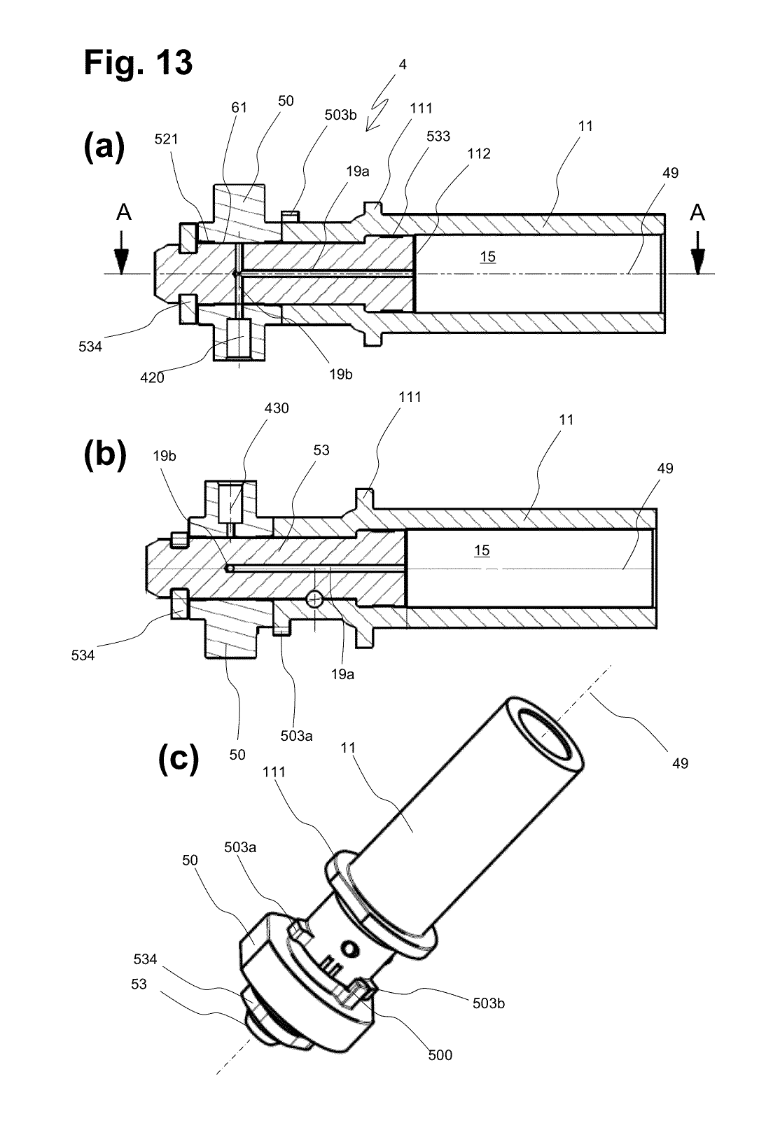

Yet another embodiment of a dosing unit 1 is depicted in FIG. 13, comprising a cylinder 11, a valve seat 53, a valve member 50, and a locking disk 534. In contrast to the embodiments discussed so far, the valve seat 53 is not an integral part of the cylinder 11, but can be within the cylinder 11. This particular variant can be assembled very easily, by introducing valve seat 53 from the back end of the cylinder 11, mounting the valve member 50, and finally the locking disk 534, thereby positively locking the four components in longitudinal direction. The valve seat 53 can sealed close the front end 112 of the cylinder 11 with a circumferential sealing 533 which at the same time can frictionally lock valve seat 53 and cylinder 11.

The valve seat 53 with attached cylinder 11 and the valve member 50 can be rotatable to each other around axis 49. The valve seat 53 can have two stoppers 503a, 503b which can interact with a cam 500 of the valve member 50, thereby limiting the rotational motion between states I and II.

The valve seat 53 can comprise a passage conduit 19 with a first portion 19a along the longitudinal axis and a perpendicular second portion 19b crossing the valve seat/core. The valve member 50 can comprise two conduits 420, 430 arranged perpendicular to the longitudinal axis 49. The function of the valve 4 can be similar to the embodiment in FIG. 9, with the faces of the valve seat 53 and the bore of the valve member 50 sealed interacting with each other. FIGS. 13(a) and (b) show the valve 4 in state I with passage conduit 19b fluidly connected to conduit 420 while conduit 430 can be sealed closed by the valve core.

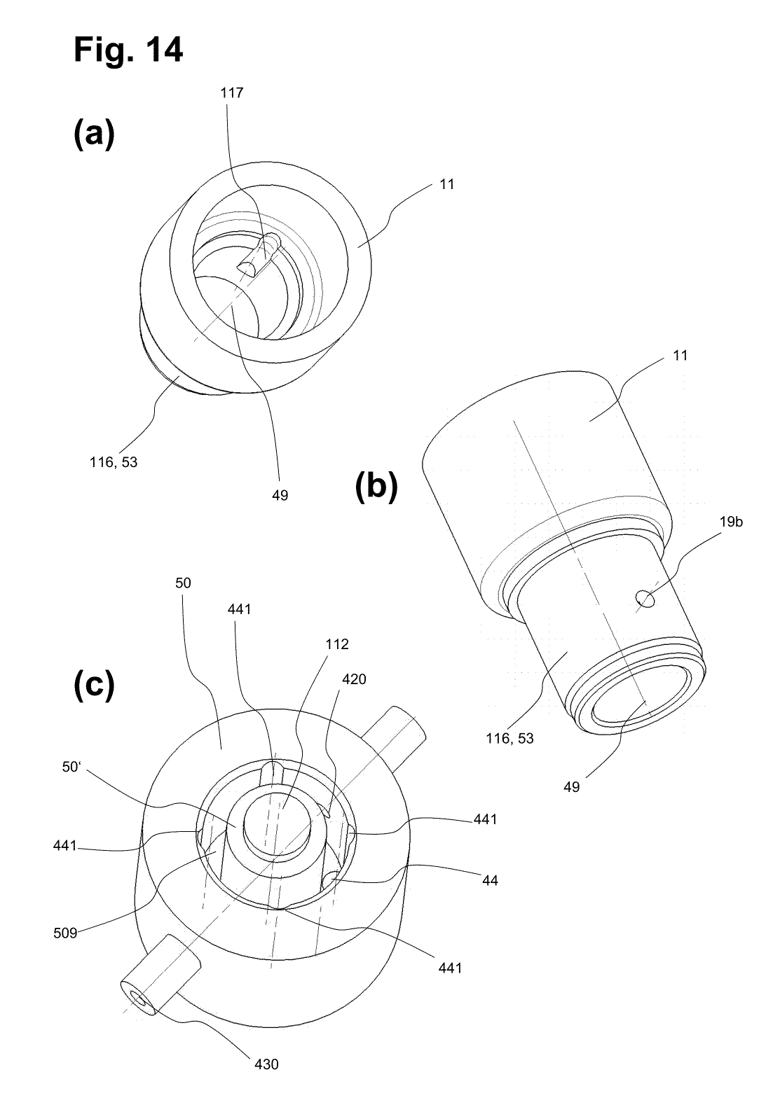

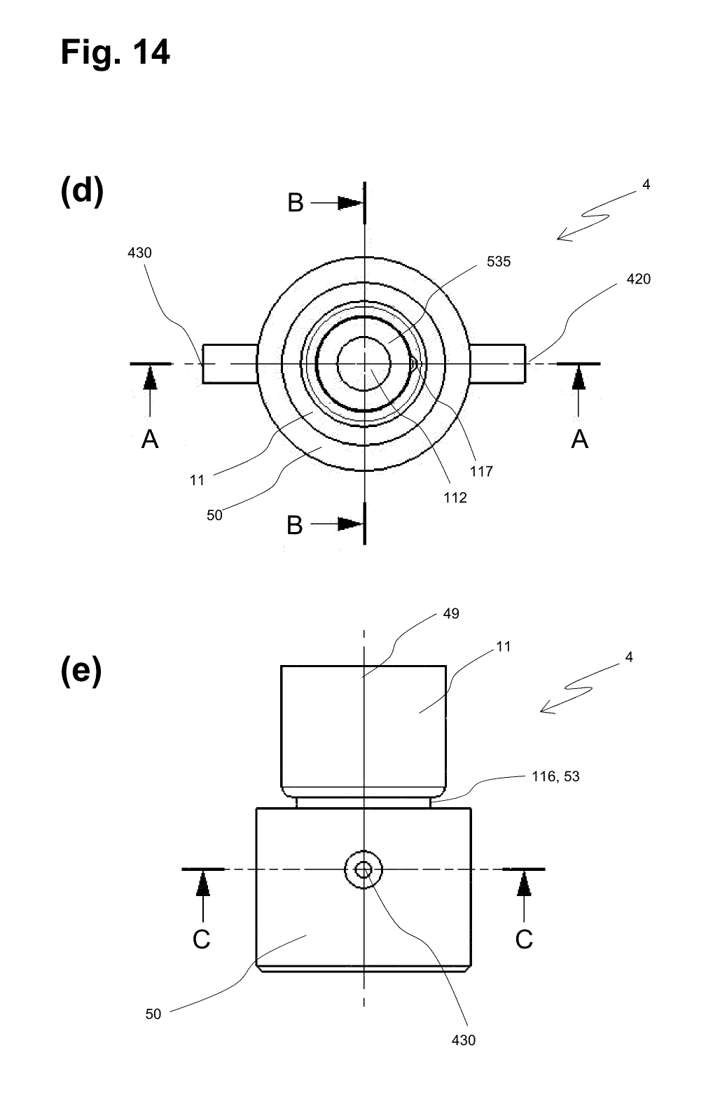

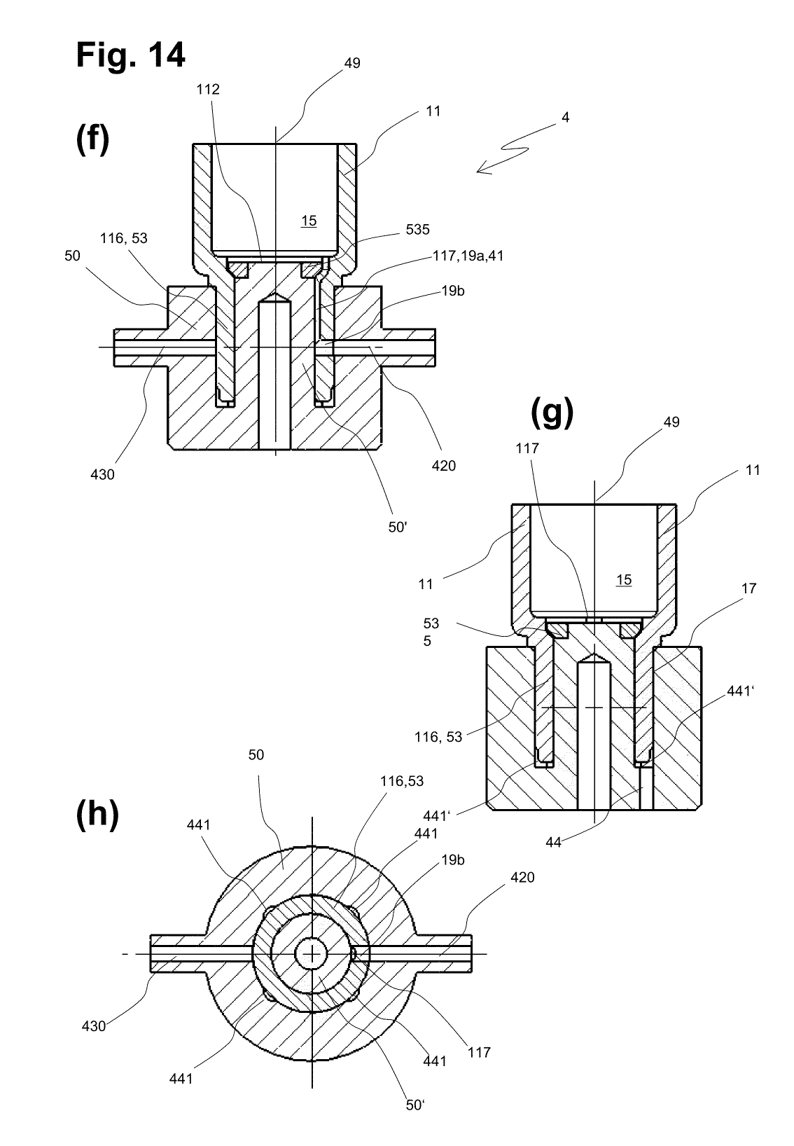

Another valve 4 is disclosed in FIGS. 14(a) to (h). FIGS. 14(a) and (b) show isometric views on the valve seat 53/cylinder 11, while FIG. 14(c) shows the valve member 50. FIGS. 14(d) to (h) show the assembled valve 4, (d) in a top view along longitudinal axis 49 with view onto the cylinder bottom 112, (e) in a side view with view onto one of the connection conduit tubes, (f) in a cross-section along plane A-A, (g) in a cross-section along plane B-B, and (h) in a cross-section along plane C-C.

A hollow cylinder 11 can comprise at its front end 112 a constricted portion 116 that can act as the valve seat 53, which can be rotatably mounted in a cylindrical bore 509 of the body of valve member 50, 50′. A circumferential shoulder of the cylinder 11 can rest on the body of valve member 50. A cylindrically shaped inner portion 50′ of the valve member can be within the cylinder 11. A locking ring 535 can be assembled, e.g. by ultrasonic welding, to the inner portion 50′ of the valve member 50 and can determine the relative axial position of valve member 50 and cylinder 11. In addition, the locking ring 535 can axially secure the cylinder 11 with respect to valve member 50. The locking ring 535 can alternatively be assembled to cylinder 11 or can be replaced by a different means, such as a snap ring.

In the embodiment, the liquid-tight connection between cylinder wall and cylinder bottom can be provided along the abutting longitudinal surfaces of the inner part 50′ of the valve member 50 and the inner side of the valve seat 53.

The restricted portion 116/valve seat 53 of the cylinder 11 can comprise a longitudinal groove 117 arranged on the inner cylindrical surface of the restricted portion which together with the outer surface of the cylindrical inner portion 50′ of the valve member 50 can sealingly form a passage conduit portion 19a opening toward the inner volume 15 of the pump cylinder 11. Two conduits 420, 430 can be radially arranged in the portion 50 of the valve member 50 on the other side of the cylinder wall. A radial bore 19b in the wall of the valve seat 53 can be provided to establish a fluid connection between passage conduit 19a/groove 117 and one of the connection conduits 420, 430, when the opening of one of the connection conduits and the bore 19b overlap, depending on the rotational orientation of valve seat 53 and valve member 50 to each other.

Four longitudinal grooves 441 can be arranged in the cylinder wall of valve member 50, which can be connected via a small circumferential gap 441′ between the restricted portion 116 and the bottom of bore 509 to a venting conduit 44.

The valve 4 in FIGS. 14(d) to (h) can be in state I, with the passage conduit 117, 19a, 19b fluidly connected to the infusion site connection conduit 420. When the valve 4 is switched to state II, clockwise or counterclockwise, depending on the configuration chosen for the valve 4, the passage conduit 19a, 117, 19b, which can act as the pump port 41, can be temporarily connected to environment (state III) whenever the horizontal bore 19b passes a groove 441. In one embodiment, the valve 4 can pass two states III when switching from state I to state II and back.

There can exist no fluid path between the infusion site connection conduit 420 and the reservoir connection conduit 430 that does not pass at least one of the grooves 441, 441′. Thus under no circumstances can liquid flow from the reservoir 21 through the valve 4 to the infusion site, since it can drain through grooves 441, 441′ toward the venting conduit 44.

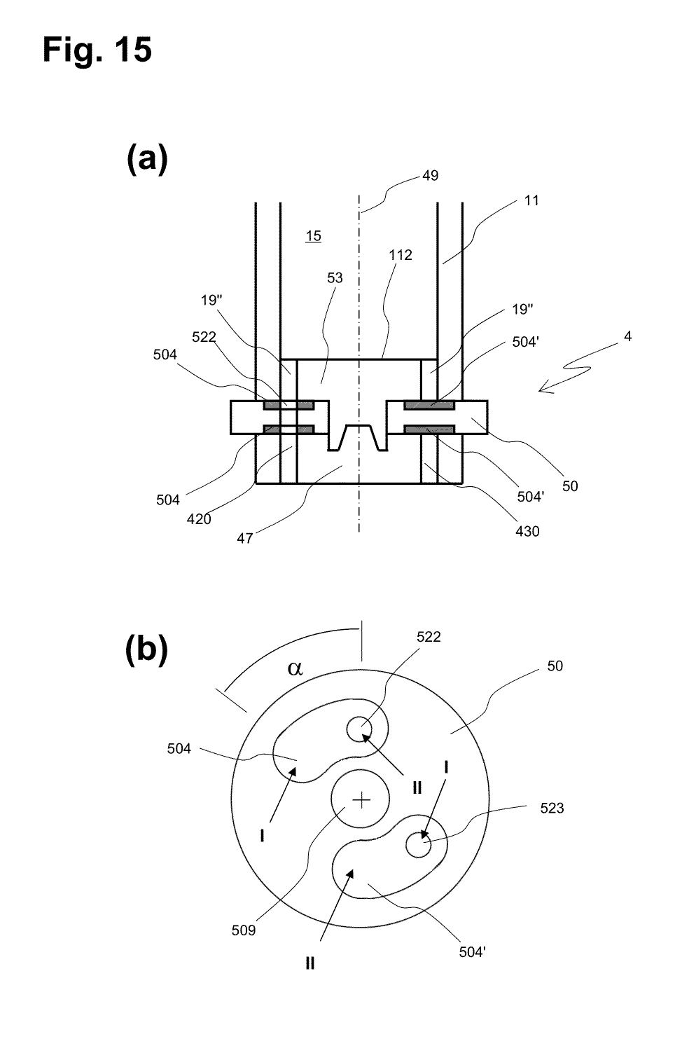

An embodiment of a dosing unit 1 with a valve member 50 in the form of a valve disk is shown in FIG. 15. A cylinder 11 with an integral valve seat 53 can be connected to bearing 47. A disk shaped valve member 50 can be rotatably mounted between valve seat 53 and bearing 47. The valve seat 53 and bearing 47 can be connected for example by a snap lock mechanism or by ultrasonic welding. The valve seat 53 can comprise two axial passage conduits. One passage conduit 19′ can be collinear with an infusion site connection conduit 420 arranged in the bearing body and the second passage conduit 19″ can be collinear with an reservoir connection conduit 430.

The valve disk 50 can comprise two sealing elements 504, 504′ made from an elastic polymer material as discussed above. Two bores 522, 523 can be located in the sealing elements, so that in a state I of the valve 4, the bore 522 in sealing element 504 can fluidly connect one passage 19′ with the conduit 420 and the other sealing element 504′ can disconnect the other passage 19″ from the conduit 430. To switch the valve 4 to state II, the cylinder 11 and valve seat 53 can be rotated in regard to the disk by an acute angle, disconnecting the first conduit 420 from the cylinder 11 and connecting the second passage 19″ to the second conduit 430.

The venting state III can be realized for example with grooves arranged on the sealing elements that are fluidly connected to a venting conduit, similar for example to the embodiment shown in FIGS. 3 to 6.

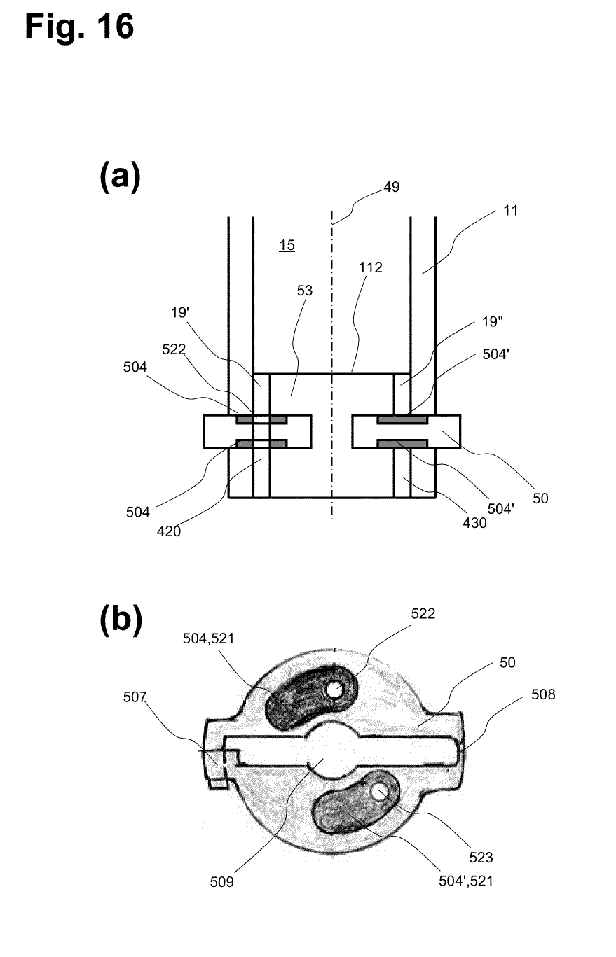

FIG. 16 depicts a variant of the valve 4 in FIG. 15(a), with no separate bearing 47. To mount the valve disk in such a valve 4, the valve disk of FIG. 16(b) can be suitable. The two half portions of the disk can be pivoted around hinge 508, for embracing the valve seat 53, and can be locked with a suitable mechanism 507 in the final conformation.

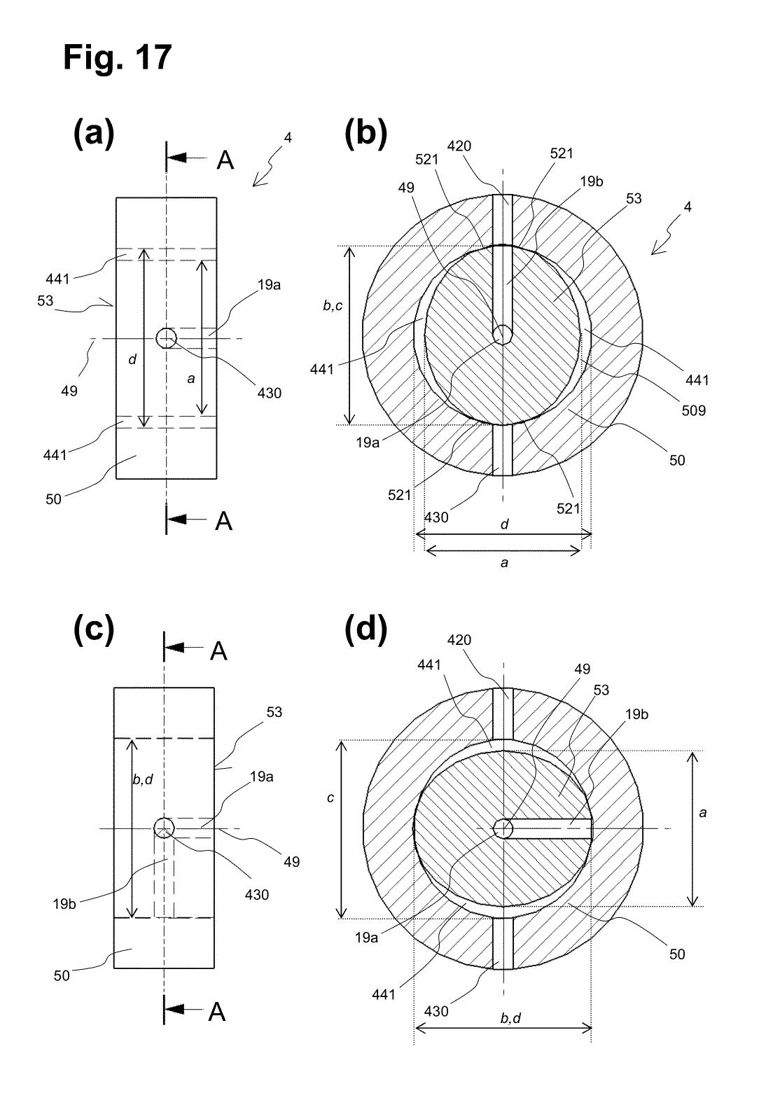

A variant of a valve 4 that can be suitable for long time storage stability prior to use is schematically depicted in FIG. 17. The basic design of this valve 4 in regard to the arrangement of the connection conduits 420, 430 and the passage conduit 19 is similar to FIGS. 9(c) and 11. The valve 4 can comprise a valve seat 53 realized as an oval-shaped, central valve core 53 oriented parallel to the longitudinal axis 49. A valve member 50 can be rotatably mounted on the valve seat 53.

The valve member 50 can have the shape of an oblate cylinder comprising the connection conduits 420, 430, and a central bore 509 for the valve core 53. The conduits 420, 430 can open toward the bore 509, at an angular distance of about 180°. The central bore 509 can have the shape of an ellipse with minor diameter c and major diameter d. Also the valve core 53 can have the shape of an ellipse, with a minor diameter a and a major diameter b. A longitudinally oriented, first portion 19a of the passage conduit within the valve core 53 can be connected to the reservoir 21 (not shown). A second portion 19b can be radially oriented along the major axis of the valve core 53.

The additional advantage of the shown embodiment of a valve 4 can be related to the ratios of different diameters a, b, c, d to each other, as will now be explained in more detail.

To avoid functional failure of the valve 4 due to material fatigue of the sealing elements of the valve 4 during storage prior to use, after manufacture the valve 4 can be in a storage mode, which is shown in FIGS. 17(c) and (d). In this storage mode, the second portion 19b of the passage conduit can be oriented in an angle of about 90° to the connection conduits 420, 430. The major diameter d of the bore 509 of the valve member 50 can be chosen equal or larger than the major diameter b of the valve core 53. Similarly the minor diameter c of the bore 509 can be chosen equal or larger than the minor diameter a of the valve core 53. As a result, in the storage mode no portion of the valve elements 50, 53 that can later interact with each other, in order to establishing a sealing connection between passage conduit 19 and connection conduits, can be mechanically stressed. Thus no material fatigue resulting from, for example, plastic flow can take place, independently from the shelf storage time of the valve 4 prior to first use.

During normal operation, the valve 4 can switch between state I and II, passing between one or two times a state III. In FIGS. 17(a) and (b), the valve 4 is in state I, with the passage conduit portion 19b being aligned to the inlet connection conduit 420 and their openings overlapping. The major diameter b of the valve core 53 can be chosen slightly larger than the minor diameter c of the bore 509. This can lead to a certain compression of the elastomeric material on the contacting surfaces of the bore 509 and the valve core 53, resulting in a sealing area 521 around the connection conduits 420, 430. The exact values for b and c can depend on the materials used for the valve elements. The connection conduit 420 can be sealed connected to passage conduit 19b, 19a and connection conduit 430 can be sealed closed.

The gap 441 between the minor axis a of the valve core 53 and the wall of the bore 509 can be fluidly connected to environment. Thus the gap 441 can act as the venting conduit 44 of the valve 4. Any possible fluid path between the two connection conduits 420, 430 can have to pass at least one of these gaps 441. Thus any liquid that may leak from the reservoir conduit 430 in case of a malfunction of the valve 4 due to, e.g., a leaking sealing, can drain through gap 441 and cannot reach connection conduit 420.

State II (not shown) can be identical to state I, except for the orientation of the valve core 53, which can be rotated by about 180° so that the passage conduit portion 19b can be aligned with connection conduit 430.

To switch the valve 4 from state I to state II, the valve core 53 can be rotated clockwise (or counterclockwise) around axis 49. During this rotation, the gaps 441 can also rotate clockwise (or counterclockwise) and can come into contact with the connection conduits 420, 430, thereby temporarily connecting the connection conduits to environment. FIGS. 17(c) and (d) shows the valve 4 halfway between state I and II with a rotation angle of the valve core of about 90°. In case the major axis d of the bore is chosen to be larger than the major axis b of the valve core, the passage conduit 19b can be connected to environment latest when reaching a rotation angle of about 90°.

In case the major axis b of the valve core is chosen to be the same as the major axis d of the bore, the passage conduit 19b cannot be connected to environment during the switching process. In such a case, the bore wall between the two connection conduit openings can be provided for example with a segmental groove that fluidly connects the passage conduit 19b in the position of FIG. 17(d) with the venting gaps 441.

Summarized one can say that for a valve 4 as described in FIG. 17, the relations can be as follows: b>c (sealing connection in state I, II); b<d (storage mode); a<c (connection conduits connected to environment). In case b=d, an additional venting path can be provided for the passage conduit.

It is noted that terms like “preferably,” “commonly,” and “typically” are not utilized herein to limit the scope of the claimed embodiments or to imply that certain features are critical, essential, or even important to the structure or function of the claimed embodiments. Rather, these terms are merely intended to highlight alternative or additional features that may or may not be utilized in a particular embodiment of the present disclosure.

Having described the present disclosure in detail and by reference to specific embodiments thereof, it will be apparent that modifications and variations are possible without departing from the scope of the disclosure defined in the appended claims. More specifically, although some aspects of the present disclosure are identified herein as preferred or particularly advantageous, it is contemplated that the present disclosure is not necessarily limited to these preferred aspects of the disclosure.

We claim:

1. A dosing unit for an infusion pump device, the dosing unit comprising:
a piston pump;
a reservoir connector fluidly connected to a reservoir;
an infusion site connector fluidly connected to an infusion set; and
a valve comprising a valve seat and a valve member that are rotatable to each other, a pump port fluidly connected to the piston pump, an upstream port fluidly connected to the reservoir connector, and a downstream port fluidly connected to the infusion site connector wherein the valve can be in a downstream state corresponding to a specific angular orientation of valve seat and valve member, where the pump port is fluidly connected to the downstream port and the upstream port is sealed closed; in a venting state corresponding to one or more specific angular orientations of valve seat and valve member where the downstream port and the upstream port are sealed closed and the pump port is fluidly connected to a venting chamber or to atmosphere; and in an upstream state corresponding to a specific angular orientation of valve seat and valve member where the pump port is fluidly connected to the upstream port and the downstream port is sealed closed and wherein upon rotation of the valve member in regard to the valve seat from the downstream state to the upstream state and/or from the upstream state to the downstream state, the valve passes at least one angular orientation of valve seat and valve member that corresponds to the venting state.

2. The dosing unit according to claim 1, wherein one or more venting passages is arranged on the valve seat and/or the valve member, the one or more venting passages being fluidly connected to the venting chamber or to atmosphere.

3. The dosing unit according to claim 2, wherein one or more of the venting passages is arranged on the valve seat and/or the valve member in such a way that any possible geometrical path between the upstream port and the downstream port in a gap formed between abutting surfaces of the valve seat and the valve member will cross at least one of the venting passages.

4. The dosing unit according to claim 2, wherein in the venting state, the pump port is fluidly connected to the one or more of the venting passages.

5. The dosing unit according to claim 1, wherein the valve passes an intermediate state where all ports are sealed closed, when switching between the upstream state and the venting state, and/or when switching between the venting state and the downstream state.

6. The dosing unit according to claim 1, wherein the valve seat comprises at least one passage conduit that is fluidly connected to the piston pump and the valve member comprises a first connection conduit fluidly connected to the infusion site connector and a second connection conduit fluidly connected to the reservoir connector, wherein the fluid connection between a passage conduit and one of the connection conduits is established when an opening of the passage conduit overlaps with an opening of the corresponding connection conduit.

7. The dosing unit according to claim 6, wherein one or more venting conduits is fluidly connectable to a passage conduit.

8. The dosing unit according to claim 1, wherein the valve seat and the valve member have limited rotational displacement in regard to each other, wherein one limit of rotational displacement corresponds to the downstream state of the valve and the other limit of rotational displacement corresponds to the upstream state of the valve.

9. The dosing unit according to claim 8, wherein the venting state of the valve corresponds to one or more rotational displacements between the two maximum displacements.

10. A dosing unit for an infusion pump device, the dosing unit comprising:
a piston pump;
a reservoir connector fluidly connected to a reservoir;
an infusion site connector fluidly connected to an infusion set; and
a valve comprising a valve seat and a valve member that are rotatable to each other, a pump port fluidly connected to the piston pump, an upstream port fluidly connected to the reservoir connector, and a downstream port fluidly connected to the infusion site connector; wherein the valve can be in a downstream state, where the pump port is fluidly connected to the downstream port and the upstream port is sealed closed; and in an upstream state, where the pump port is fluidly connected to the upstream port and the downstream port is sealed closed; wherein one or more venting passages are arranged on the valve seat and/or the valve member, the one or more venting passages are fluidly connected to a venting chamber or to atmosphere, and arranged in such a way that any possible geometrical path between the upstream port and the downstream port in a gap formed between abutting surfaces of the valve seat and the valve member will cross at least one of the venting passages.

11. The dosing unit according to claim 10, wherein the valve can be in a downstream state, where the pump port is fluidly connected to the downstream port and the upstream port is sealed closed; in a venting state where the downstream port and the upstream port are sealed closed and the pump port is fluidly connected to a venting chamber or to atmosphere; and in an upstream state where the pump port is fluidly connected to the upstream port and the downstream port is sealed closed and wherein upon rotation of the valve member in regard to the valve seat from the downstream state to the upstream state and/or from the upstream state to the downstream state, the valve passes at least one angular orientation of valve seat and valve member that corresponds to the venting state.

12. The dosing unit according to claim 11, wherein the valve passes an intermediate state where all ports are sealed closed, when switching between the upstream state and the venting state, and/or when switching between the venting state and the downstream state.

13. The dosing unit according to claim 12, wherein in the venting state, the pump port is fluidly connected to one or more of the venting passages.

14. The dosing unit according to claim 10, wherein the valve seat comprises at least one passage conduit that is fluidly connected to the piston pump, and the valve member comprises a first connection conduit fluidly connected to the infusion site connector and a second connection conduit fluidly connected to the reservoir connector and wherein the fluid connection between a passage conduit and one of the connection conduits is established when an opening of the passage conduit overlaps with an opening of the corresponding connection conduit.

15. The dosing unit according to claim 14, wherein one or more venting conduits is fluidly connectable to a passage conduit.

16. The dosing unit according to claim 10, wherein the valve seat and the valve member have limited rotational displacement in regard to each other, wherein one limit of rotational displacement corresponds to the downstream state of the valve and the other limit of rotational displacement corresponds to the upstream state of the valve.

17. The dosing unit according to claim 16, wherein the venting state of the valve corresponds to one or more rotational displacements between the two maximum displacements.

18. The dosing unit according to claims 10, wherein either the valve seat or the valve member of the valve is an integral part of a cylinder of the piston pump.

19. The dosing unit according to claim 18, wherein the rotation axis of the valve seat and the valve member is collinear to a longitudinal axis of the cylinder.

20. An infusion pump device with a dosing unit according to claim 10.

21. The infusion pump device according to claim 20, wherein the reservoir is fluidly connected to the reservoir connector of the dosing unit and/or an infusion set is fluidly connected to the infusion site connector of the dosing unit.

22. A method for safely conveying a liquid medication in an infusion pump device, the method comprising:
a) providing an infusion pump device with a dosing unit according to claim 1, with a reservoir fluidly connected to the reservoir connector of the dosing unit and/or an infusion set fluidly connected to the infusion site connector of the dosing unit;
b) switching the valve of the dosing unit to the venting state;
c) switching the valve of the dosing unit to the upstream state;
d) conveying an amount of liquid medication from the reservoir to the pump cylinder of the piston pump by generating a negative pressure in the pump cylinder;
e) switching the valve of the dosing unit to the venting state;
f) switching the valve of the dosing unit to the downstream state;
g) conveying a certain amount of liquid medication, in one or more portions, from the pump cylinder to the infusion site connector by generating a positive pressure in the pump cylinder; and
h) repeating steps b) to g).
